(19) United States
(12) Patent Application Publication
(10) Pub. No.: US 2008/0067271 A1
(43) Pub. Date: Mar. 20, 2008

(54) Apparatus for mixing and/or crushing substances into fine particles and method of crushing substances into fine particles using such apparatus

(76) Inventor: Sukeyoshi Sekine, Shizuoka (JP)

Correspondence Address: WENDEROTH, LIND & PONACK, L.L.P., 2033 K STREET N. W., SUITE 800, WASHINGTON, DC, 20006-1021, US

(21) Appl. No.: 11/892,467

(22) Filed: Aug. 23, 2007

Related U.S. Application Data
(63) Continuation of application No. 11/415,282, filed on May 2, 2006, now abandoned.
Application No. 11/415,282 is a continuation of application No. 10/476,892, filed on Nov. 6, 2003, now abandoned.

(30) Foreign Application Priority Data
May 7, 2001 (JP) 2001-136229

(51) Int. Cl. B02C 17/06

(52) U.S. Cl. 241/92

(57) ABSTRACT

An apparatus for mixing and/or crushing substances into fine particles is disclosed, wherein the apparatus includes a fluid flow path that is formed by a first structural member having a plurality of cells thereon, each having its front side open, and a second structural member having a plurality of cells thereon, each having its front side open. The first structural member and the second structural member are arranged adjacently each other such that the open front sides of the cells on one structural member face opposite the open front sides of corresponding cells on the other structural member, with the cells on the one structural member being placed in alternate positions with regard to the corresponding cells on the other structural member, and with each of the cells on the one structural member communicating with at least one or more of the cells on the other structural member. When a right-angled isosceles triangle is given and when segments connecting any arbitrary point on two sides and any arbitrary point on a perpendicular from a vertex to a base is given, each of the cells has a shape defining an area that is equal to one half an area of the right-angled isosceles triangle that is obtained by turning the above segments clockwise and counter-clockwise about the arbitrary point on a corresponding side as an origin to intersect these segments with the base, and then by connecting these segments. A method for mixing and/or crushing substances into fine particles is also disclosed, wherein the method comprises steps performed by using the above-described apparatus wherein, a fluid, in which substances to be crushed are mixed, is introduced into an inlet of a cylindrical casing under applied pressure, and then subjected to a mixing and/or crushing process while flowing the fluid through the fluid flow path.

This application is a continuation of U.S. application Ser. No. 11/415,282, filed May 2, 2006, which is a continuation of U.S. application Ser. No. 10/476,892, filed Nov. 6, 2003, which was the National Stage of International Application No. PCT/JP02/00175, filed Jan. 15, 2002.

### BACKGROUND

1. Technical Field

The present invention relates to an apparatus for mixing various substances into a fluid, an apparatus for crushing various substances into fine particles, an apparatus for mixing and crushing various substances into fine particles, and a method of crushing various substances into fine particles using any of such apparatuses. More particularly, the present invention provides such apparatuses and methods wherein substances can be mixed and/or crushed into fine particles without having to use mechanical power.

Any of the apparatuses and methods according to the present invention may be utilized in applications, such as those involving a process of crushing foodstuffs and raw medicinal substances into fine particles, a process of deactivating/sterilizing any enzymes/spores contained in the foodstuffs and raw medicinal substances, a process of deodorizing the substances, a process of rendering industrial wastes harmless, and any other like processes.

2. Prior Art

A typical conventional stationary-type mixing apparatus that can be operated without having to use mechanical power is disclosed in Japanese patent application as published under No. S58 (1983)-13822. This stationary mixing apparatus is shown in FIG. 59(a) through FIG. 59(c), and comprises a cylindrical casing 203 having an inlet 201 on one end and an outlet 202 on an opposite end, and a plurality of fluid conducting units provided inside the cylindrical casing 203. Each of the fluid conducting units includes a larger-diameter round plate 206 and a smaller-diameter round plate 207 that are arranged coaxially such that they are placed adjacently each other. In each fluid conducting unit, the larger-diameter round plate 206 and smaller-diameter round plate 207 have a plurality of polygonal (multi-sided) cells 204, 205 on respective sides facing opposite each other. The cells 204, 205 on each of the round plates 206, 207 are arranged like a honeycomb, and each of the cells 204, 205 has its front side open.

The larger-diameter round plate 206 has a diameter that matches an inner diameter of the cylindrical casing 203, and has a fluid flow hole 208 through a center thereof.

The larger-diameter round plate 206 and smaller-diameter round plate 207 are arranged such that the open front sides of the cells 204 on the larger-diameter round plate 206 are located to face opposite the open front sides of corresponding cells 205 on the smaller-diameter round plate 207, with the cells 204 on the larger-diameter round plate 206 being placed in alternate positions with regard to corresponding cells 205 on the smaller-diameter round plate 207, and with each of the cells on the one round plate, that is, the larger or smaller round plate, being capable of communicating with at least one or more of the cells on the other round plate, that is, the other of the smaller and larger round plate. Thus, each individual fluid conducting unit may include one such larger-diameter round plate 206 and one such smaller-diameter round plate 207 that are disposed to face opposite each other.

It is shown in FIG. 59(a) that a number of such individual fluid conducting units are installed within the cylindrical casing 203, wherein in any two adjacent fluid conducting units, the round plates of the same diameter, that is, the larger-diameter round plates or smaller-diameter round plates, are placed to face opposite each other.

There is installed one fluid conducting unit on each of the opposite ends of the cylindrical casing 203, and each of the fluid conducting units on the opposite ends has its larger-diameter round plate located on a respective end side, so that it can communicate with the inlet 201 or outlet 202 through respective center fluid flow hole 208.

Substances being processed, such as being mixed, usually have the form of a fluid, which may be introduced under applied pressure through the inlet 201 into a space inside the cylindrical casing 203. The fluid may then flow through the fluid flow hole 208 in the first fluid conducting unit located on an upstream side, flowing into the cylindrical casing 203. The fluid that has passed through the larger-diameter round plate 206 in the first fluid conducting unit may flow toward the smaller-diameter round plate 207 in the first fluid conducting unit, where the fluid is prevented from flowing in a straight direction. When passing through the cells 205, 204 on the smaller-diameter round plates 205, 204 in the first and second fluid conducting units that are located adjacently each other, the fluid must thus change its direction, going radially from a center toward an outer circumferential side. Then, the fluid goes toward the smaller-diameter round plate 207 in the third fluid conducting unit located downstream of the second fluid conducting unit, where the fluid passes through a gap between the smaller-diameter round plate 207 and an inner circumferential wall of the cylindrical casing 203, flowing toward the cells 205 on the smaller-diameter round plate 207 in a fourth fluid conducting unit located downstream of the third fluid conducting unit and then flowing toward the larger-diameter round plate 206 in the fourth fluid conducting unit where the fluid is prevented from flowing in a straight direction, changing its direction. Then, the fluid passes through the cells 205, 204 in the third and fourth fluid conducting units that communicate with each other, flowing from an outer circumferential side toward the center. Then, the fluid passes through the fluid flow hole 208 in the larger-diameter round plate on the fourth fluid conducting unit, flowing toward the cells 204 on the larger-diameter round plate 206 in a fifth fluid conducting unit located downstream of the fourth fluid conducting unit. This is repeated until the fluid reaches a final fluid conducting unit, where the fluid flows out of the cylindrical casing 203 through the outlet 202.

It may be understood from the above description that when the fluid passes through the cells 204 on the larger-diameter round plates in the adjacent fluid conducting units that face opposite each other and then through the cells 205 on the smaller-diameter round plates on the adjacent fluid conducting units that face opposite each other, these cells may have an effect of dispersing, reversing or swirling a flow of the fluid. That is, the fluid may be diffused or dispersed, flowing radially from the center toward the outer circumferential side and from the outer circumferential side toward the center. This flow may be repeated alternately until the fluid can be mixed completely. In this way, a mixing process can occur efficiently.

In the prior art mixing apparatus described above, a plurality of polygonal (multi-sided) cells 204, 205 are provided like a honeycomb on each of the larger and smaller round plates, but those cells 204a, 205a that are located on respective outer peripheral sides have polygonal shapes that are not similar to those of the cells 204, 205; that is, one or two sides deviate from the polygonal shape. When the fluid flows through those cells having the shape deviating from the polygonal shape, the flow may tend to be focused upon those cells. This means that the fluid would only flow through those cells. Accordingly, all possible routes through which the fluid can flow will be lost. This may be referred to as the “short path” effect. Thus, the fluid might collide with those cell walls, causing the fluid to have complex dispersing, reversing, swirling, radially dispersing and focusing actions, which may be repeated. This would reduce a mixing efficiency. Problems that have been described so far are encountered by a stationary mixing apparatus disclosed in Japanese patent application No. 58 (1983)-133822.

For a round plate having a circular shape such as the one that is disclosed in Japanese patent application No. 58 (1983)-133822, it is very difficult to arrange the cells on any two adjacent round plates opposite each other such that the cells on one round plate can be placed in alternate positions with regard to the cells on the other round plate, with each of the cells on the one round plate being able to communicate with at least one or more of the cells on the other round plate. In this case, it may be possible to provide guide pins for arranging the cells in appropriate positions, but some of the cells must be eliminated because of presence of the guide pins. This means that a number of the cells must be reduced. As described above, this might also cause the fluid to collide with the cell walls, causing its flow to have the complex dispersing, reversing, swirling, radially dispersing, and focusing actions. Repeating these actions would affect the mixing efficiency.

It should be noted that the round plate 206 such as the one that is employed in the Japanese patent application No. 58(1983)-133822 has its outer diameter that matches an inner diameter of the casing 203 so that the round plate can provide a sealing function. This makes it difficult to move the round plate 206 into or out of the casing 203. To avoid this, the inner diameter of the casing 203 may be larger by a thickness of a seal, thereby blocking flow of fluid. The casing 203 must be long enough to accommodate a plurality of fluid conducting units, and each of the fluid conducting units must be sealed along an entire length of the casing 203. If pressure under which the fluid is supplied is increased, it may break some of the seal, thereby creating a partial gap between the outer diameter of the round plate 206 and the inner diameter of the casing 203. If such situation occurs, the fluid may take the short path through the gap along the entire length of the casing 203, flowing toward the outlet 202 without undergoing a required mixing action, and the fluid may leave the casing 203 through the outlet 202. This would prevent a uniform mixing efficiency.

It may be appreciated that the stationary mixing apparatus as disclosed in the Japanese patent application No. 58(1983)-133822 is only designed to mix substances. Thus, the apparatus cannot provide capabilities of crushing substances into fine particles or improving a quality of the substances as well as mixing the substances.

For example, there is a conventional method of crushing a fluid of particular substances into ultrafine particles having a particle size of between about 1 nm and 0.1 μm, wherein any ingredient extracting element and raw substances may be evaporated by thermal plasma or the like into a particular gas within a reactor vessel, from which ultrafine particles may be generated by causing the raw substances to react with the gas. According to this method, the ultrafine particles may be obtained by performance of a long-time, continuous process in which the gas that contains the ultrafine particles in a floating and dispersing state within a reactor vessel may be introduced into a recovery vessel through a heat exchanger equipped with a cooling pipe where the ultrafine particles may be cooled by a coolant, and the ultrafine particles within the recovery vessel may be passed through a filter and collected.

The apparatus that may be used in conjunction with the above method includes the reactor vessel and recovery vessel for implementing the method, and therefore must have a larger construction. The apparatus can be running continuously for a relative long time, but the ultrafine particles that have been collected into the recovery vessel by passing them through the filter will have produced secondary and tertiary coagulations as some time elapses. Thus, the ultrafine particles will gather into gross particles, or will have a temperature that exceeds a heat resistant temperature tolerance of machine components that are located upstream of the recovery vessel. When this occurs, illumination of the thermal plasma must be suspended for a certain time so that the fine particles can be recovered and the machine components can be disassembled and cleaned. Then, the machine components must be reassembled. This process is laborious and inefficient.

There is also another conventional apparatus and method. According to this apparatus and method, a circulating fluid that contains any ingredient extracting element and raw substances being processed may be jetted into a storage tank through a jet nozzle, allowing resulting atomized particles in the storage tank to flow by inertial action through a fluid flow path that is placed under a low pressure, and allowing fine particles in the atomized particles to flow through the fluid flow path, and then to be attracted through an outlet of the fluid flow path into a particle capturing device that is placed under low pressure reduced by a decompressor pump. The fine particles that have been captured- into the particle capturing device under the low pressure may expand themselves, and therefore must be classified into different sizes by allowing the fine particles to pass through primary and secondary filters in a gross particle capturing device, and must then be captured by the gross particle capturing device.

The apparatus that may be used in conjunction with the method described above must have a large construction accordingly. A jet nozzle may become clogged with a mixture of finely crushed particle substances and liquid (circulating fluid). Each time the jet nozzle becomes clogged, machine components must be disassembled, cleaned and reassembled. As the apparatus is only designed to provide particular fine particles that are desired to be obtained, supply of the raw substances being crushed into fine particles must be increased. Thus, wastes that may result from an ingredient extracting process will also be increased. Disposal of these wastes will create an environmental problem.

There are also several conventional methods for promoting or retarding a dissolving process of some environment polluting substances such as substances that are difficult to dissolve, and progress of a chemical reaction of such substances with any gaseous reacting substances or solid reacting substances, or for promoting or retarding a generating and dissolving process of some chemical substances by controlling the chemical reaction. Some methods take advantage of ultra-critical processing, and other methods take advantage of electromagnetic waves, ultrasonic waves, infrared rays, far infrared rays and the like.

For a method that takes advantage of ultra-critical processing, substances that are subject to the ultra-critical processing may be crushed into fine particles, which may then be mixed into a fluid. Then, the fluid may be introduced into a reactor vessel that is placed under the ultra-critical conditions by increasing an internal temperature and pressure up to a particular numerical level. Then, any gaseous oxidizer or liquid oxidizer such as air, oxygen, carbon dioxide and the like is forced into the reactor vessel where an oxidizing dissolution reaction or other actions can occur.

Dissolving substances being dissolved and their chemical reaction with any reaction substances may essentially consist of dissolving the substances by causing their molecules to collide with each other and then mixing them together. Thus, the substances must first be introduced into respective reactor vessels, where they must then be mixed together.

It should be appreciated, however, that when the apparatus is placed under the ultra-critical conditions in which an internal pressure is set to a high level, problems may occur with power required to drive the apparatus for mixing or stirring the substances fed into the apparatus, or on seals provided in the reactor vessel. An apparatus that may be used in conjunction with the above method must have a large construction, and desired processing cannot be performed satisfactorily.

In the method that takes advantage of electromagnetic waves for dissolving substances that are hard to dissolve, raw substances may be introduced into the reactor vessel that is heated by induction, where a reaction and dissolving process for the substances may be promoted by heating the substances by induction heating as well as by electromagnetic wave heating performed by electromagnetic waves that are produced by the induction heating.

For a method that takes advantage of ultrasonic waves for dissolving substances hard to be dissolved, any liquid substance and any gaseous substance being processed may be fed into the reactor vessel, where those substances may be exposed to ultrasonic waves coming from an ultrasonic wave source in the reactor vessel so that optimal mixing conditions or quick reaction speed can be obtained.

For a method that takes advantage of infrared rays or far infrared rays for generating and/or dissolving any chemical substances, a chemical reaction may be controlled by illuminating the infrared rays or far infrared rays into the reactor vessel so that a generation and dissolving process for the substances can be promoted or retarded.

### SUMMARY OF THE INVENTION

In light of the problems associated with the prior art that has been described above, the present invention proposes to provide apparatuses and methods that will be described below in specific details.

One object of the present invention is to provide a mixing apparatus that provides a uniform mixing capability that has been enhanced by adding an appropriate improvement to the prior art stationary mixing apparatus described above.

Another object of the present invention is to provide a mixing apparatus that adds an improvement to the prior art stationary mixing apparatus described above, so that it can contain machine components that are easy to be assembled, including a cylindrical casing having an inner surface that can be worked with ease, and can thus reduce total manufacturing or assembling costs.

A further object of the present invention is to provide a mixing apparatus that adds an improvement to the prior art stationary mixing apparatus described above, so that it can prevent any possible fluid leaks that would occur if any partial gap is present, and can also prevent non-uniform mixing that would occur if fluid takes a shortcut path (short path).

Still another object of the present invention is to provide a mixing apparatus that adds an improvement to the prior art stationary mixing apparatus described above, such that it can provide mixing and/or crushing capabilities for crushing substances such as foodstuffs and medicinal substances, or even substances that are hard to dissolve such as environment polluting substances, into finely crushed spherical particles having particle sizes of between about 1 nm and 1 μm.

There is no mixing apparatus that has a comparatively small size, and despite its small size, is capable of producing and/or creating fine particles by allowing a fluid to flow under applied pressure through a fluid flow path formed by cells each having its front side open.

In view of the above fact, it is a further object of the present invention to provide a mixing apparatus that adds an improvement to the prior art stationary mixing apparatus described above, such that despite its small size, the apparatus can provide mixing and/crushing capabilities for crushing substances into fine particles, more specifically, crushing various types of fibrous substance into fine particles.

Any of the apparatuses provided by the present invention may be used as a heat exchanger, and may also be used to dissolve such substances or promote a chemical reaction for those substances by taking advantage of a critical process, ultra-critical process, electromagnetic waves, ultrasonic waves, infrared rays, far infrared rays and the like.

The present invention also proposes to provide several methods that may be implemented by any of the mixing apparatuses mentioned above for crushing substances into fine particles. Any of the methods proposed by the present invention may be used in conjunction with the critical process or ultra-critical process or other processes, such as substance dissolving, chemical reaction promoting and the like, that take advantage of electromagnetic waves, ultrasonic waves, infrared rays, far infrared rays and the like.

The processes, such as the substance dissolving, chemical reaction promoting and like processes, which may be implemented by the methods of the present invention by taking advantage of the electromagnetic waves, ultrasonic waves, infrared rays, far infrared rays and the like may provide desired processing results, such as improved substance surface activity, improved substance quality, promoted chemical promotion and improved substance dissolving.

Any of the apparatuses and methods provided by the present invention permits crushing, reaction promotion, dissolving and mixing processes for substances to occur continuously so that consistent processing results can be obtained, without having to use a large scale machine.

In order to accomplish the above objects, the apparatus according to the present invention is designed to mix and/or crush substances into fine particles, and comprises a cylindrical casing having a hollow portion therein and having an inlet on one end and an outlet on an opposite end, wherein a fluid flow path is provided to run between the inlet and outlet through the cylindrical casing. The fluid flow path has unique structural features that will be described below.

First, the fluid flow path may be formed by a first structural member having a plurality of first cells thereon, each having its front side open, and a second structural member having a plurality of second cells thereon, each having its front side open, wherein the first structural member and second structural member are arranged adjacently each other such that the first cells on the first structural member and second cells on the second structural member have respective open front sides placed to face opposite each other and in alternate positions with regard to each other, with each of the cells on one structural member communicating with at least one or more of the cells on the other structural member.

The open front side of each of the first cells and second cells has a shape that may be defined by a shape surrounded by figure P-S-Q-S2-R-S1-P, wherein when an imaginary right-angled isosceles triangle ABC, with A referring to a vertex and AC, BC referring to two equal sides, is given: S refers to any arbitrary point other than points A, R that is located along segment A-R connecting vertex A and midpoint R on base B-C; P refers to any arbitrary point other than points A, B that is located along side A-B; Q refers to any arbitrary point other than points A, C that is located along side A-C; S1 refers to a point where segment P-S will intersect with the base B-C when the segment P-S is turned about point P; and S2 refers to a point where segment Q-S will intersect with the base B-C when the segment Q-S is turned about point Q.

When the fluid flow path is formed by the first structural member having a plurality of first cells thereon, each having its front side open, and the second structural member having a plurality of second cells thereon, each having its front side open, wherein the first structural member and second structural member are arranged adjacently each other such that the first cells on the first structural member and second cells on the second structural member have respective open front sides placed to face opposite each other and in alternate positions with regard to each other, with each of the cells on one structural member communicating with at least one or more of the cells on the other structural member, a space on the front sides of the cells facing opposite each other may be divided by at least one or more cells on the other structural member. Then, an area and volume where the space is divided on the open front sides of the cells are different before and after the fluid flow path, respectively. Specifically, the fluid flow path is formed by a plurality of structural members such that any two adjacent structural members are arranged opposite each other, with the open front sides of the cells on one structural member facing opposite the open front sides of the cells on the other structural member, with the cells on the one and the other structural members being arranged in alternate positions with regard to each other, and with each of the cells on the one structural member communicating with at least one or more of the cells on the other structural member. The fluid flow path runs from the inlet toward the outlet of the cylindrical casing, and includes a plurality of continuous divisional sections whose areas and volumes are different before and after a location where each divisional section exists along a continuous fluid flow path. When a fluid of substances being processed, such as being mixed and/or being crushed, is fed into the fluid flow path, the fluid may cause colliding, reversing, swirling, radial dispersing, and gathering motions each time the fluid passes continuously through the divisional sections each having a different area and volume. These motions may be repeated until the fluid reaches an end of the fluid flow path. More specifically, when the fluid passes through one divisional section having an area and volume different from those of another divisional section, on one hand, the fluid may undergo a coagulation action that is increased by an embracing pressure. When the fluid passes through one divisional section having an area and volume different from those of another divisional section, on the other hand, the fluid may be released from the embracing pressure at once, and may then undergo dissolving and crushing actions. The fluid can be mixed uniformly and/or crushed into very fine particles having a desired size, such as nearly pure spherical forms, by repeating the above motions and actions.

The apparatus according to the present invention includes the first structural member having a plurality of first cells thereon, each having its open front side formed like a particular shape described above, and the second structural member having a plurality of second cells thereon, each having its open front side formed like the particular shape described above, wherein the fluid flow path is formed by the first and second structural members that are arranged adjacently each other, with the cells on the first structural member facing opposite the cells on the second structural member, with the first cells being placed in alternate positions with regard to the second cells facing opposite the first cells, and with each of the cells on one structural member communicating with at least one or more of the cells on the other structural member, and wherein the fluid flow path runs through the cylindrical casing from the inlet toward outlet of the cylindrical casing. Substances that have been crushed into gross particles, as well as liquid, may be mixed into a fluid, and the fluid may be introduced under applied pressure into the apparatus described above in accordance with the present invention. When the fluid is traveling through the apparatus, pressures may be produced inside and outside the fluid, such as a primary embracing pressure, secondary embracing pressure, exploded dispersion, twisting, swelling, kneading and friction. Thus, these gross particle substances may be crushed into ultrafine particles and molecules. In addition, surface activity improvement, quality improvement, reaction promotion and dissolving can be provided for the ultrafine particles and molecules in a consistent and continuous manner.

In the above description, it should be noted that the segments P-S and Q-S may be formed as a single straight line, or a broken line consisting of several straight lines. These line segments may also be formed as a sine curve, arc curve and the like. Otherwise, these segments may be formed as a combination of a straight line, broken line and curved line.

It should also be noted that the shape of the open front side for the first and second cells, each having its front side open, may be determined by setting point P and point Q as a midpoint along the side A-B and a midpoint along the side A-C, respectively.

An area of the shape surrounded by figure P-S-Q-S2-S1-P thus obtained may correspond to one half an area of the imaginary original right-angled isosceles triangle.

This may be explained more clearly by referring to FIG. 1. In FIG. 1, point P and point Q may be located at a midpoint along side A-B and at a midpoint along side A-C, respectively. Then, points where respective perpendiculars dropped from point P and point Q intersect with base B-C are designated as S3 and S4, respectively. In this case, an area of rectangle P-S3-R-S4-Q-P may correspond to one half an area of right-angled isosceles triangle ABC.

It may be appreciated from the above description that point S1 is a point where segment P-S will intersect with the base B-C when the segment P-S is turned about point P, and point S2 is a point where segment Q-S will intersect with the base B-C when the segment Q-S is turned about point Q. It may be seen from FIG. 1 that the area of the portion denoted by S5 in FIG. 1 will be equal to the area of the portion denoted by S6, and the area of the portion denoted by S7 will be equal to the area of the portion denoted by S8. As a result, the area of the shape surrounded by figure P-S-Q-S2-S1-P drawn as described above will be equal to one half the area of the imaginary original right-angled isosceles triangle ABC.

In the apparatus described above, the shape of the open front side for the first cells and second cells, each having its front end open, may be determined by setting points P and Q at the midpoint along the side A-B and at the midpoint along the side A-C. Thus, the fluid flow path may be formed by a continuous sequence of divisional sections, each of which has an area and volume that are different from those of any other divisional spaces before and after this divisional section. When a fluid of substances being mixed and/or crushed is fed under applied pressure into the fluid flow path, passing through a fluid flow path, the fluid may undergo a uniform mixing process and/or may undergo a crushing process in which substances may be crushed into fine particles that have desired spherical forms, such as nearly pure spherical forms. In addition to this effect, other advantageous effects may be obtained as described below.

As described above, the shape of the open front side for the first cells and second cells may be defined by placing points P and Q on the midpoint along the side A-B and on the midpoint along the side A-C, respectively, with the first structural member having the plurality of first cells thereon, each having its front side open, and the second structural member having the plurality of second cells thereon, each having its front side open, and with the first cells and second cells being arranged on respective entire surfaces thereof without any gaps being produced between any adjacent cells, respectively.

FIGS. 1 through 13, FIG. 31(a), FIG. 32(a), FIG. 33(a), and FIG. 53 represent examples of shapes for the open front sides of the first and second cells that are arranged on the first and second structural members, respectively, in which each of the cells has a right-angled isosceles triangle as a basic shape as defined above.

It may be seen from these figures that each of the first cells and second cells having its front side open has a portion that is surrounded by a shape surrounded by figure P-S-Q-S2-S1-P and a shape that is similar to, but is smaller or larger than, the above shape, and that portion may be raised as a wall on the first structural member and second structural member, respectively. FIG. 31(b), FIG. 32(b) and FIG. 33(b) represent examples of the first cells and second cells, each having its front side open, in which each cell has a portion that is surrounded by a shape surrounded by figure P-S-Q-S2-S1-P and the shape that is similar to, but is smaller or larger than, the above shape, and that portion may be raised as a wall on the first structural member and second structural member, respectively.

It may be understood from the foregoing description that the area of the shape surrounded by the figure P-S-Q-S2-S1-P should be equal to one half the area of the imaginary original right-angled isosceles triangle ABC. In this way, the first structural member having the plurality of first cells thereon, each having its front side open, and the second structural member having the plurality of second cells thereon, each having its front side open, can have the first cells and second cells arranged over entire surfaces thereof, respectively, without any gaps being produced between any adjacent cells. If the above conditions are met, the segment S2-S1 in the figure P-S-Q-S2-S1-P does not have to be a straight line segment on the base B-C.

In the apparatus of the present invention described above, for example, the shape of the open front side for the first and second cells, each having its front side open, may also be defined by placing point P and point Q on a point symmetrical location with the segment A-R as a center on the sides A-B, A-C, respectively, and by making either the segment S2-R or R-S1 in the segment S2-R-S1 into another segment having any shape different from a straight line segment on the base B-C, and then making the segment as point symmetrical with regard to the segment having the above any shape into another segment, with midpoint R as the center.

FIG. 14 represents an example of the above case, in which point P and point Q are midpoints on sides A-B and A-C, respectively. In this example, the area of the portion denoted by S9 will be equal to the area of the portion denoted by S10. Thus, the shape of the open front side for the first cells and second cells, each having its front side open, can be defined as efficiently as the case where point P and point Q are the midpoints on the sides A-B and A-C, respectively.

In the apparatus of the present invention described above, it should be noted that the fluid flow path may be formed in the axial direction of the cylindrical casing or in a direction perpendicular to the axial direction of the cylindrical casing. For example, the fluid flow path may be provided in the axial direction of the cylindrical casing, if the sides on which the open front sides for the plurality of first cells on the first structural member and the open front sides for the plurality of second cells on the second structural member face opposite each other extend in the axial direction of the cylindrical casing, while the fluid flow path may be provided in the direction perpendicular to the axial direction of the cylindrical casing, if the sides opposite each other extend in the direction perpendicular to the axial direction of the cylindrical casing.

For example, the first structural member and second structural member may be mountable within the cylindrical casing, and the fluid flow path may be provided in the axial direction of the cylindrical casing or in the direction perpendicular to the axial direction of the cylindrical casing. It should be understood that there are several ways in which the first structural member and second structural member may be mountable within the cylindrical casing. For example, one way is to provide recesses around an inner circumferential wall of the cylindrical casing, and construct the first and second structural members so that they can be fitted in these recesses. When the cylindrical casing and the first and second structural members are constructed in this way, it would become easier to work the inner circumferential wall of the cylindrical casing, and assemble the cylindrical casing, first structural member and second structural member into the complete apparatus. This construction provides an advantage in that it ensures that no partial gaps would occur between the cylindrical casing and the first structural member and second structural member. That is, the short path would be prevented.

As a variation of the apparatus of the present invention described above, the cylindrical casing may be formed by the first structural member so that the plurality of first cells, each having its front side open, can be provided on an inner circumferential wall of the first structural member forming the cylindrical casing, and the plurality of second cells, each having its front side open, can be provided on an outer circumferential wall of the second structural member that may be mounted inside the first structural member forming the cylindrical casing. In this way, the fluid flow path may be provided in the axial direction of the cylindrical casing formed by the first structural member. As an alternative form of the fluid flow path, which is formed by placing the first and second structural members such that the cells on the first structural member can face opposite the cells on the second structural member face, the fluid flow path may be formed by permitting the second structural member having a particular outer circumferential diameter to be fitted into the cylindrical casing formed by the first structural member having an inner circumferential diameter that matches the outer circumferential diameter of the second structural member. For example, the fluid flow path may be formed by permitting the second structural member simply to be fitted in the recess provided on the cylindrical casing formed by the first structural member. In this variation, the first structural member forming the cylindrical casing and the second structural members can be worked easily so that they can provide an inner circumferential surface and outer circumferential surface, respectively, and that these members can be assembled into a complete machine. This variation may also provide an advantage in that it ensures that no partial gaps would occur between the cylindrical casing and the first structural member and second structural member. That is, the short path would be prevented.

As a further variation of the above variation, the first structural member forming the cylindrical casing may have a divisible construction, and mounting and removing of the second structural member within and from the cylindrical casing may be performed by dividing the cylindrical casing. This makes it easy to mount the second structural member within the cylindrical casing formed by the first structural member, and assemble those members into a complete machine. This may also provide easy maintenance of those members. It may be appreciated that the cylindrical casing may have the divisible construction or form that permits the cylindrical casing to be divided in an axial direction thereof.

As a variation of the apparatus described above, the first structural member and the second structural member may comprise a first plate member having a plurality of cells, each having its front side open, on one side thereof, or a second plate member having a plurality of cells, each having its front side open, on both of opposite sides thereof, wherein the first and second plate members are mounted inside the cylindrical casing so that the fluid flow path can be formed in the axial direction of the cylindrical casing or in the direction perpendicular to the axial direction of the cylindrical casing. For example, recesses may be provided on the inner circumferential wall of the cylindrical casing, and the first and second plate members may have the form or construction that permits the first and second plate members to be fitted in the recesses on the cylindrical casing. Then, the fluid flow path may be formed between the first and second plate members that are placed to overlap each other tightly. This makes it easy to work the cylindrical casing formed by the first plate member so that it can provide an inner circumferential surface, and to assemble these members into a complete machine. This may also provide an advantage in that it ensures that no partial gaps would occur between the cylindrical casing and the first plate member and second plate member. That is, the short path would be prevented.

It should be noted that the plurality of cells, each having its front side open, on another side of the second plate member may be provided by rotating the cells on the one side of the second plate member through a predetermined angle, such as 45 degrees, 90 degrees, or 180 degrees at locations of the plurality of cells, each having its front side open, on the other side of the second plate member corresponding to locations of the plurality of cells, each having its front side open, on the one side of the second plate member.

As one alternative form, the plurality of cells, each having its front side open, on the other side of the second plate member may be provided at locations of the cells on the other side of the second plate member that are different from locations of the plurality of cells, each having its front side open, on the one side of the second plate member.

As a further variation, the plurality of cells, each having its front side open, on the other side of the second plate member may be provided by rotating the cells on the one side of the second plate member through a predetermined angle, such as 45 degrees, 90 degrees, or 180 degrees at locations of the plurality of cells, each having its front side open, on the other side of the second plate member that are different from locations of the plurality of cells, each having its front side open, on the one side of the second plate member.

In any of the forms described above, when the first and structural members are placed to adjoin each other tightly such that the cells, each having its front side open, on the first structural member can face opposite the cells, each having its front side open, on the second structural member, or when the two structural members are placed to adjoin each other tightly such that the cells on the one and other structural members can face opposite each other, the cells on the one structural member can be placed in alternate positions with regard to the cells on the other structural member, and each cell on the one structural member can communicate with at least one or more of the cells on the other structural member.

As described above and as shown in FIGS. 1 through 14, each of the cells has a right-angled isosceles triangle as its basic shape, and those cells are arranged such that their respective open front sides face opposite each other, with the cells facing opposite each other being placed in alternate positions, and each of the cells on one structural member can communicate with at least one or more of the cells on the other structural member. When the fluid flow path is thus formed, the space that may be created on the open front sides of those cells can have many different configurations and forms (area, volume). When the fluid flows through the fluid flow path, it can manifest more complicated motions that are caused by colliding, dispersing, reversing and swirling actions of a fluid. Thus, a mixing and/or crushing process can be promoted.

As a variation of the apparatus described above, a plurality of frame members may be provided on upstream and downstream sides of a location where the fluid flow path is formed by the first and second structural members inside the cylindrical casing, wherein these frame members have openings arranged like a honeycomb and communicating with each other in the axial direction of the cylindrical casing, and are placed to overlap each other so that the openings on the adjacent frame members facing opposite each other can be placed in alternate positions with regard to each other in the direction perpendicular to the axial direction of the cylindrical casing.

In the above variation, when fluid flows through the fluid flow path in the cylindrical casing, it can have complicated motions that are caused by the flow path formed by the openings of the frame members before and after the fluid flow path, as well as complicated motions that are caused by colliding, dispersing, reversing and swirling actions of the fluid. A mixing and/or crushing process can be promoted further.

In this variation, the cylindrical casing may also have a divisible construction. For example, it may be divisible in the axial direction thereof, and the first structural member including the first plate member and the second structural member including the second plate member may be mounted inside the cylindrical casing and removed from the cylindrical casing, by dividing the cylindrical casing.

In the embodiment in which the frame members are employed, the cylindrical casing may have a divisible construction, for example, it may be divided into two parts in the axial direction thereof, and the first structural member including the first plate member and the second structural member including the second plate member, as well as the first and second frame members arranged to overlap each other, may be mounted inside the cylindrical casing and removed from the cylindrical casing by dividing the cylindrical casing into the two parts.

This embodiment permits the first and second plate members as well as the frame members to be mounted inside the cylindrical casing with ease, making it easy to assemble, disassemble, and reassemble these members for maintenance purposes.

In the apparatus described above in accordance with the various embodiments of the present invention, the plurality of first cells, each having its front side open, on the first structural member and the plurality of second cells, each having its front side open, on the second structural member should desirably have an identical shape. This provides an advantage in that when the first structural member and second structural member are arranged such that the cells, each having its front side open, on the first structural member and the cells, each having its front side open, on the second structural member can face opposite each other, by using a simple way of rotating the first cells through an angle of 45 degrees, 90 degrees or other degrees, at locations of the second cells on the second structural member that correspond to locations of the first cells on the first structural member and then providing the second cells on the second structural member, the cells facing opposite each other can be placed in alternate positions with regard to each other, with each of the cells on one structural member communicating with at least one or more of the cells on the other structural member. This may also provide an advantage in that the first cells and the corresponding second cells can have a regular size, which prevents the short path from occurring.

In the apparatus described above in accordance with the various embodiments of the present invention, the plurality of first cells, each having its front side open, on the first structural member and/or the plurality of second cells, each having its front side open, on the second structural member may be arranged at any location on the first and second structural members, respectively, as long as the first and second structural members are arranged such that they can form the fluid flow path as described above. It should be noted, however, that the fluid flow path may also be formed by arranging the first cells and second cells like a honeycomb on the first and second structural members, respectively.

In this honeycomb arrangement, a shape of the open front side for the first and second cells can be defined by placing point P and point Q on the midpoints along the sides A-B and A-C of the imaginary right-angled isosceles triangle, respectively. In this way, the first and second cells can be arranged on an entire surface of any side of the first and second structural members, respectively, without producing any gaps between the adjacent cells.

In the apparatus described above in accordance with the various embodiments of the present invention, an inlet space and an outlet space may be provided on an upstream side and downstream side of a location where the fluid flow path is formed by the first and second structural members, wherein the inlet space has a conical shape whose diameter is increasing from the inlet in a downstream direction, and the outlet space has a conical shape whose diameter is decreasing toward the outlet. This variation may provide an advantage in that when fluid is flowing through the cylindrical casing, it can have motions, such as dispersing, concentrating and colliding, before and after the fluid flow path, as well as complicated motions, such as colliding, dispersing, reversing and swirling, which are caused as the fluid flows through the fluid flow path. Thus, a mixing and/or crushing process can be promoted.

In this case, it should be noted that when the fluid flow path between the inlet space and outlet space, each having a conical shape, is being formed between an inner circumferential wall of the inlet space and an outer circumferential wall of the outlet space, corresponding structural members having the conical shape may be disposed in the inlet space and outlet space.

As a variation of the apparatus described above in accordance with the various embodiments of the present invention, the first structural member may be the cylindrical casing, and the plurality of first cells, each having its front side open, which are provided on the first structural member in accordance with the previous embodiments may be provided on an inner circumferential wall of the cylindrical casing, and the plurality of second cells, each having its front side open, which are provided on the second structural member in accordance with the previous embodiments may be provided on an outer circumferential wall of the structural member that is mounted inside the cylindrical casing. In this variation, the fluid flow path may be formed between the inner circumferential wall of the cylindrical casing and the outer circumferential wall of the structural member mounted inside the cylindrical casing. In the manner described above, when the fluid flow path between the inlet space and outlet space, each having a conical shape, is being formed between the inner circumferential wall of the inlet space and the outer circumferential wall of the outlet space, the corresponding structural members having the conical shape may be disposed in the inlet space and outlet space. This provides an advantage in that the fluid can flow smoothly through the fluid flow path formed between the inner circumferential wall and the outer circumferential wall of the structural member mounted inside the cylindrical casing.

In any of the embodiments of the apparatus according to the present invention that have been described so far, the first structural member and/or second structural member may be made of any material selected from the group consisting of carbons or composite metal materials such as combinations of carbon and other metals, ceramics, and other minerals.

The cylindrical casing may also be made of any material selected from the group consisting of carbons or composite metal materials such as combinations of carbon and other metals, ceramics, and other minerals.

The first structural member and/or second structural member may also be made of any one of natural resins and synthetic resins.

Similarly, the cylindrical casing may also be made of any one of natural resins and synthetic resins.

The first structural member, second structural member and cylindrical casing may also be made of any of thermal conductive materials such as copper, aluminum, carbon and the like, in which case an apparatus including such components may also be used as a heat exchanger.

It should be appreciated that the first structural member, second structural member and cylindrical casing may also be made of any of SUS metals.

In any of the embodiments of the apparatus according to the present invention that have been described so far, magnets may be disposed on an outer circumferential wall of the cylindrical casing.

The cylindrical casing may have any cross sectional shape, such as round, elliptical, and polygonal (triangle, square, and the like). The cylindrical casing may include a central portion that has any cross sectional shape, such as round, elliptical, and polygonal (triangle, square, and the like), and portions having conical and pyramidal shapes that are provided before and after the inlet and outlet sides, respectively.

In any of the embodiments of the apparatus according to the present invention that have been described so far, the apparatus may be used in conjunction with any one of an ultrasonic wave illuminating device, electromagnetic wave illuminating device, high-frequency wave illuminating device, and laser beam illuminating device that may be linked to an upstream and/or downstream side of the apparatus.

In any of the embodiments of the apparatus according to the present invention that have been described so far, the apparatus may include an inlet that may be linked to an upstream and/or downstream side of an apparatus for feeding some particular types of agents that contain oxygen or alkali agents.

Those agents that are fed through this inlet have an action or effect to cause an oxidization reaction of substances being processed, or to neutralize any acid generating ingredients that may be contained in the substances being processed.

It may be appreciated that the apparatus according to any of the embodiments described so far permits different types of substances to be dissolved and/or crushed into ultrafine particle levels or molecular levels although the apparatus has a small-size construction.

When the above mentioned devices are linked to the upstream and/or downstream side of the apparatus, the apparatus may be used as a mixing/separating reaction continuous generator.

When an electromagnetic wave illuminating device, infrared ray illuminating device, ultrasonic wave illuminating device or the like is linked to the downstream side of the apparatus, or is also linked to the upstream side if required, a process of dissolving environment polluting substances and substances hard to be dissolved can be improved remarkably, and a process of generating and dissolving chemical substances can also be improved remarkably.

A method proposed in accordance with one aspect of the present invention for crushing substances into fine particles may be used in conjunction with any of the apparatuses described so far, wherein a fluid in which substances to be processed (crushed) are mixed is introduced under applied pressure through its inlet, and can undergo a fine particle crushing process while flowing from the inlet toward the outlet of the cylindrical casing of the apparatus.

A method proposed in accordance with another aspect of the present invention for crushing substances into fine particles may be used in conjunction with any of the apparatuses described so far, wherein a fluid in which substances to be processed (crushed) are mixed is introduced under applied pressure through its inlet, and can undergo a fine particle crushing process under continuous critical condition or ultra-critical condition while flowing continuously from the inlet toward the outlet of the cylindrical casing of the apparatus.

As it is used in this specification, the term “critical condition” should be understood to mean that when a particular substance is placed at a temperature higher than a critical temperature of the substance and under a pressure higher than a critical pressure of the substance, the substance is in a gas-liquid coexisting state, not simply in a gaseous state nor in a liquid state. As it is used in this specification, the term “ultra-critical condition” should be understood to mean that a particular substance is placed in a state in which it is impossible to tell whether it is in a gaseous state or a liquid state when it is above the critical condition. The critical temperature and critical pressure are constant values that are specific to particular types of substances.

Both the critical condition and ultra-critical condition for a particular type of substance being processed can be attained, for example, by admixing any gas phase oxidizer or liquid phase oxidizer such as air, oxygen and carbon dioxide with the substance in the form of a fluid, feeding this resulting mixture continuously under an applied pressure into the cylindrical casing so that the fluid can flow from the inlet toward the outlet of the cylindrical casing, placing the fluid under the critical condition or ultra-critical condition specific to the particular type of substance, and bringing the fluid into an appropriate critical condition or ultra-critical condition. It should be noted that pressure may be controlled by adjusting applied pressure under which the fluid is fed into the cylindrical casing, and temperature may be controlled by adjusting a temperature at which the cylindrical casing is heated.

In the method and apparatus according to one aspect of the present invention described above, particular industrial wastes, for example, may be crushed into particles in the form of a fluid. Then, the fluid may be introduced under applied pressure into the cylindrical casing together with a gas of pure oxygen, and molecules that are bonded with each other may be dissolved, reduced harmless and discharged from the outlet.

In the method and apparatus according to another aspect of the present invention described above, a mixture of particular substances and a solvent such as carbon dioxide in the form of a fluid may be introduced continuously under applied pressure into the apparatus, or the cylindrical casing, and may be crushed into fine particles, dissolved, or have its quality improved by placing the fluid continuously under an appropriate critical condition or ultra-critical condition.

By using the method and apparatus of the present invention in conjunction with any one of an electromagnetic wave illuminating device, infrared ray illuminating device, far infrared ray illuminating means, and laser beam illuminating device that may be provided on the upstream and/or downstream sides of the cylindrical casing, a process of crushing substances into fine particles, dissolving the substances and improving quality can be performed more efficiently.

According to the method and apparatus of the present invention, a process of dissolving and improving quality for substances hard to dissolve, harmful organic substances, environment polluting substances, chemical substances and the like can be performed efficiently.

In summary, the apparatus for mixing and/or crushing substances into fine particles in accordance with the various embodiments of the present invention includes a fluid flow path, wherein the fluid flow path is formed by arranging a plurality of first cells, each having its front side open, and a plurality of second cells, each having its front side open, such that the open front sides of the first cells and the open front sides of the second cells face opposite each other, with the first cells being placed in alternate positions with regard to the second cells and with each of the first cells communicating with at least one or more of the second cells. When a right-angled isosceles triangle having two sides, a vertex and base is given, the open front side of each of the first and second cells may be formed by a basic geometrical shape having an area that is equal to one half an area of the right-angled isosceles triangle that may be formed by turning segments, connecting arbitrary points along the two sides and an arbitrary point along a perpendicular dropped from the vertex to the base, clockwise and counter-clockwise about the arbitrary points on the sides as origins, and intersecting these segments with the base. The fluid flow path thus formed may include a continuous sequence of divisional sections, each having an area and volume that are different from those of each adjacent divisional section before and after the adjacent divisional section. Then, a fluid of substances being processed may be fed under applied pressure into the fluid flow path, and may experience internal and external pressures continuously, such as primary embracing pressure, secondary embracing pressure, exploded dispersion, twisting, swelling, kneading, friction and the like, while the fluid is flowing through a continuous sequence of divisional sections having the area and volume different from those of each adjacent divisional section. A process of crushing substances into ultrafine particles and molecules, dissolving substances, and improving quality of substances can proceed in this way.

By using the apparatus and method of the present invention in conjunction with an electromagnetic wave illuminating device, ultrasonic wave illuminating device, infrared ray illuminating device and the like, a process of dissolving substances hard to dissolve, controlling a chemical reaction for chemical substances to generate chemical substances, promoting or retarding a dissolving process, improving surface activity, improving quality and the like can be performed efficiently.

### BRIEF DESCRIPTION OF THE DRAWINGS

FIG. 1 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 2 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 3 is a diagram that illustrates how a shape of a cell having one front side open is defined so that the cell can be formed on a surface of a structural member in order to provide a fluid flow path in a apparatus for mixing and/or crushing substances into fine particles in accordance with one embodiment of the present invention;

FIG. 4 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 5 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 6 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 7 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 8 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 9 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 10 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 11 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 12 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 13 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 14 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIGS. 15(a) and 15(b) illustrate an apparatus for mixing and/or crushing substances into fine particles in accordance with one embodiment of the present invention, in which FIG. 15(a) represents a cross sectional view with some non-critical parts not being shown, and FIG. 15(b) represents a side elevational of FIG. 15(a);

FIGS. 16(a) and 16(b) illustrate the apparatus shown in FIG. 15(a), in which FIG. 16(a) represents an enlarged plan view showing a first plate member provided in the apparatus, and FIG. 16(b) represents an enlarged plan view showing a second plate member provided in the apparatus;

FIGS. 17(a) and 17(b) illustrate the first and second plate members, in which FIG. 17(a) represents a cross section taken along line A-A in FIG. 16(a), and FIG. 17(b) represents a cross section taken along line B-B in FIG. 16(b);

FIG. 18 represents a perspective view of the first plate member shown in FIG. 16(a):

FIG. 19 represents a perspective vies of the second plate member shown in FIG. 16(b);

FIG. 20 represents a perspective view illustrating how the first plate member and the second plate member are placed one over the other;

FIGS. 21(a) and 21(b) illustrate the first and second plate members that are placed one over the other, in which FIG. 21(a) represents a plan view illustrating how the first and second plate members are placed one over the other, and FIG. 21(b) represents a cross section taken along line C-C in FIG. 21(a);

FIG. 22 represents a perspective view illustrating an outlook of the apparatus for mixing and/or crushing substances into fine particles shown in FIG. 15(a) in accordance with one embodiment of the present invention;

FIG. 23 represents an exploded perspective view illustrating how components of an apparatus for mixing and/or crushing substances into fine particles are assembled together in accordance with one embodiment of the present invention;

FIGS. 24(a)-24(h) illustrate components of the apparatus for mixing and/or crushing substances into fine particles shown in FIG. 23, in which FIG. 24(a) represents a front elevation of a cylindrical casing with a covering mounted thereon, FIG. 24(b) represents a side elevation of FIG. 24(a), FIG. 24(c) represents a cross sectional view illustrating an internal structure of the cylindrical casing with some non-critical parts not being shown, FIG. 24(d) represents a side elevation of FIG. 24(c), FIG. 24(e) represents a front elevation illustrating how plate members are fitted inside the cylindrical casing, FIG. 24(f) represents a side elevation of FIG. 24(e), FIG. 24(g) represents a cross section of the covering, and FIG. 24(h) represents a side elevation of FIG. 24(g);

FIG. 25 represents a cross sectional diagram illustrating how a fluid flows in the apparatus shown in FIG. 23 for mixing and/or crushing substances into fine particles, with some non-critical parts not being shown;

FIG. 26 is a partly enlarged view of FIG. 25;

FIGS. 27(a) and 27(b) illustrate an apparatus for mixing and/or crushing substances into fine particles in accordance with one embodiment of the present invention, in which FIG. 27(a) represents a cross section with some non-critical parts not being shown, and FIG. 27(b) represents a side elevation of FIG. 27(a);

FIGS. 28(a)-28(c) illustrate the apparatus for mixing and/or crushing substances into fine particles shown in FIGS. 27(a) and 27(b), in which FIG. 28(a) represents a front elevation of a structural member that is fitted inside the cylindrical casing of the apparatus with some non-critical parts not being shown, FIG. 28(b) represents a side elevation of FIG. 28(a), and FIG. 28(c) represents a cross section taken along line D-D in FIG. 28(a);

FIGS. 29(a) and 29(b) illustrate how components of the apparatus for mixing and/or crushing substances into fine particles shown in FIGS. 27(a) and 27(b) are assembled together, in which FIG. 29(a) represents a front elevation of a structural member fitted inside a cylindrical casing with some non-critical parts not being shown, and FIG. 29(b) represents a front elevation of the cylindrical casing with some non-critical parts not being shown;

FIG. 30 is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention;

FIG. 31(a) is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention, and FIG. 31(b) is a perspective view of the cell having the shape defined in FIG. 31(a);

FIG. 32(a) is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention, and FIG. 32(b) is a perspective view of the cell having the shape defined in FIG. 32(a);

FIG. 33(a) is a diagram that illustrates how a shape of a cell having one front side open is defined so that the cell can be formed on a surface of a structural member in order to provide a fluid flow path in an apparatus for mixing and/or crushing substances into fine particles in accordance with one embodiment of the present invention, and FIG. 33(b) is a perspective view of the cell having the shape defined in FIG. 33(a);

FIG. 34(a) is a plan view of a plate member having cells thereon, each of which has its front side open and has the shape defined in FIGS. 31(a) and 31(b), and FIG. 34(b) is a side elevational view of FIG. 34(a);

FIG. 35(a) is a plan view of a plate member having cells on both sides thereof, each of which has its front side open and has the shape defined in FIGS. 31(a) and 31(b), and FIG. 35(b) is a side elevational view of FIG. 35(a);

FIG. 36(a) is a cross sectional view of the apparatus for mixing and/or crushing substances into fine particles having a fluid flow path formed by the plate members shown in FIGS. 35(a) and 35(b) in accordance with one embodiment of the present invention, and FIG. 36(b) is a side elevational view of FIG. 36(a);

FIG. 37 is a diagram that illustrates how fluid flow occurs through a flow path in the apparatus shown in FIG. 36;

FIG. 38 presents a microscopic picture of a fluid that has been obtained before experimental testing is performed by using the apparatus shown in FIG. 36;

FIG. 39 presents the microscopic picture of FIG. 38 as it is magnified;

FIG. 40 presents a microscopic picture of a fluid that has been obtained one minute after the experimental testing was performed by using the apparatus shown in FIG. 36;

FIG. 41 presents a microscopic picture of a fluid that has been obtained three minutes after the experimental testing was performed by using the apparatus shown in FIG. 36;

FIG. 42 presents a microscopic picture of a fluid that has been obtained five minutes after the experimental testing was performed by using the apparatus shown in FIG. 36;

FIGS. 43(a) and 43(b) show an apparatus for mixing and/or crushing substances into fine particles in accordance with one embodiment of the present invention, in which FIG. 43(a) represents a front view and FIG. 43(b) represents a side elevational view;

FIG. 44 is a schematic diagram illustrating how two units of the apparatus of FIG. 43(a) are connected with each other;

FIG. 45(a) represents a cross section of inlet and outlet sides of the apparatus of FIG. 43(a) with some non-critical parts not being shown, and FIG. 45(b) represents a cross section of a central portion of the apparatus of FIG. 43(a) with some non-critical parts not being shown;

FIG. 46(a) is a plan view illustrating how frame members are mounted inside the apparatus shown in FIG. 43(a), and FIG. 46(b) is a plan view illustrating how structural members are also mounted inside this apparatus;

FIG. 47(a) is a front elevational view illustrating how fluid flow paths are formed by frame members inside the apparatus shown in FIG. 43(a), and FIG. 47(b) is a front elevational view illustrating how fluid flow paths are also formed by structural members inside this apparatus;

FIG. 48(a) is a plan view showing a first plate member employed in the apparatus shown in FIG. 43(a), FIG. 48(b) represents a cross section of the first plate member taken along line E-E of FIG. 48(a), and FIG. 48(c) represents a perspective view of the first plate member;

FIG. 49(a) is a plan view showing a second plate member employed in the apparatus shown in FIG. 43(a), FIG. 49(b) represents a cross section of the second plate member taken along line F-F of FIG. 49(a), and FIG. 49(c) represents a perspective view of the second plate member;

FIG. 50(a) is a plan view illustrating how frame members are placed one over the other inside the apparatus shown in FIG. 43(a), FIG. 50(b) represents a cross section of the frame members taken along line G-G of FIG. 50(a), and FIG. 50(c) represents a perspective view of the frame members;

FIGS. 51(a) and 51(b) illustrate how a fluid flows through the apparatus shown in FIG. 43(a) so that the fluid can be mixed and/or crushed into fine particles in accordance with different embodiments of the present invention, in which FIG. 51(a) represents a cross section according to a first embodiment, and FIG. 51(b) represents a cross section according to a second embodiment;

FIG. 52 is a front perspective view of an apparatus for mixing and/or crushing substances into fine particles in accordance with one embodiment of the present invention;

FIG. 53 is a diagram that illustrates how a shape of a cell having one front side open is defined so that the cell can be formed on a surface of a structure in order to provide a fluid flow path in accordance with the embodiment of FIG. 52;

FIG. 54(a) is a front elevational view illustrating a cylindrical casing in the apparatus shown in FIG. 52, FIG. 54(b) is an exploded view of components that make up an individual fluid conducting unit in the apparatus of FIG. 52, and FIG. 54(c) is a front elevational view illustrating how individual fluid conducting units are coupled together in the apparatus of FIG. 52;

FIG. 55 is a perspective view illustrating how a fluid flows through the apparatus of FIG. 52 so that the fluid can be mixed and/or crushed into fine particles;

FIG. 56 is a front elevational view illustrating how soybeans are crushed into very fine particles in the apparatus of FIG. 52 in accordance with one embodiment of the present invention;

FIGS. 57(a)-57(c) are block diagrams showing methods for mixing and/or crushing substances into fine particles in accordance with different embodiments of the present invention that may be employed in the apparatus of the present invention, in which FIG. 57(a) is a block diagram of a method according to a first embodiment, FIG. 57(b) is a block diagram of a method according to a second embodiment, and FIG. 57(c) is a block diagram of a method according to a third embodiment;

FIG. 58 is a block diagram showing a continuous ultra-critical process of crushing plastic wastes into fine particles that may take place in the apparatus of the present invention in accordance with one embodiment of the present invention; and

FIGS. 59(a)-59(c) are schematic diagrams illustrating operation of a typical conventional stationary mixing apparatus, in which FIG. 59(a) represents a cross section of the apparatus, FIG. 59(b) represents a perspective view of a larger-diameter round plate provided in the apparatus, and FIG. 59(c) represents a perspective view of a smaller-diameter round plate provided in the apparatus.

### DETAILED DESCRIPTION OF THE PREFERRED EMBODIMENTS

Now, the present invention will be described in further detail with reference to several preferred embodiments of the present invention by referring to the accompanying drawings. It should be understood that arrangements, shapes and positional relationships of machine components, elements and parts that will be described below by referring to the accompanying drawings are only given in general terms that help understand a concept of the present invention. It should also be understood that specific values and compositions (material types) of the machine components, elements and parts are only given by way of examples. It should be appreciated, therefore, that the present invention is not limited to any of the embodiments that will be described below, and should only be understood from the technical scope of the invention as defined in the appended claims and may be modified in various ways and manners.

### Embodiment 1

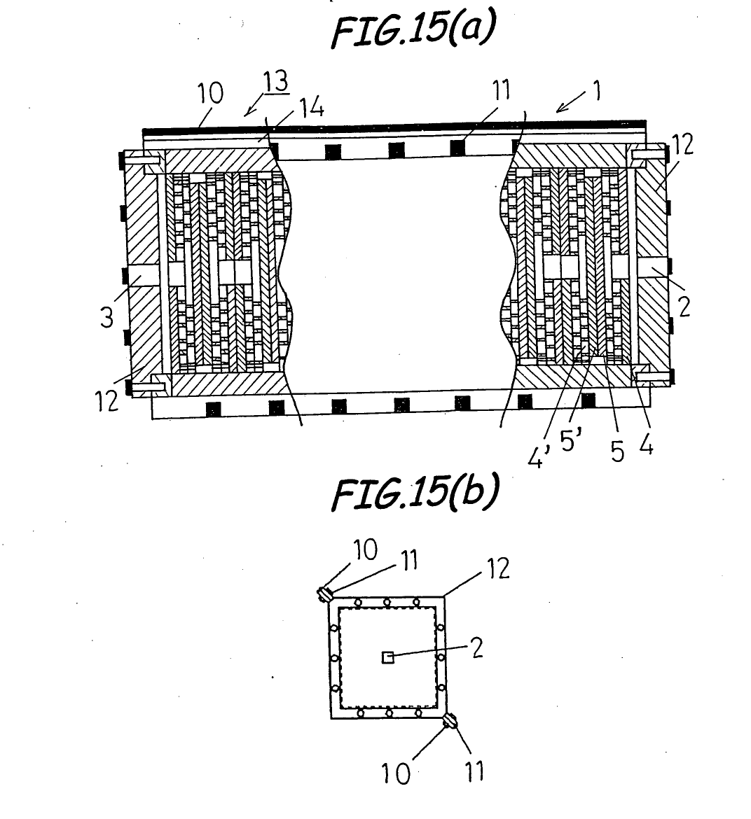

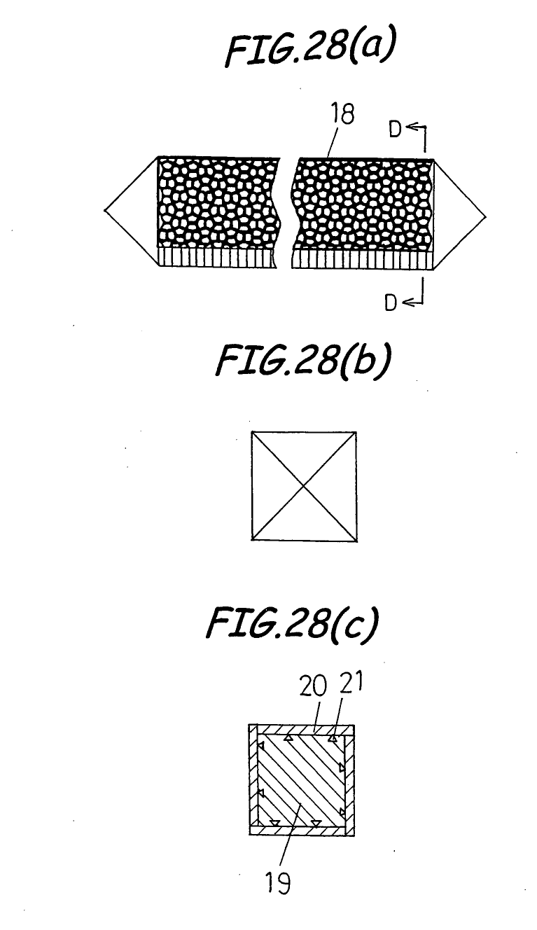

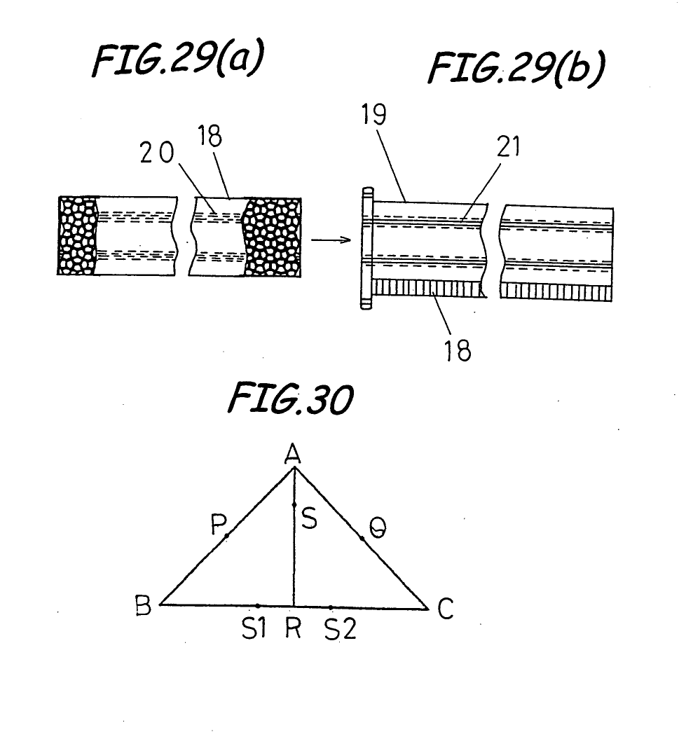

Referring first to FIGS. 15(a) through FIG. 29(b), a first embodiment of the present invention is described.

FIG. 15(a) is a schematic diagram that represents a cross section of an apparatus for mixing and/or crushing substances into fine particles. As shown in FIG. 15(a), the apparatus comprises a cylindrical casing I that has a hollow portion therein, and has an inlet 2 on one end and an outlet 3 on an opposite end. The cylindrical casing 1 further includes a fluid flow path that is formed by a first structural member and a second structural member.

In the following description, the apparatus for mixing and/or crushing substances into fine particles may be referred to simply as the apparatus for convenience of simplicity, but it should be understood that this means the apparatus for mixing and/or crushing substances into fine particles, except otherwise specified.

The first and second structural members includes a plurality of first plate members 4 and a plurality of second plate members 5, respectively, which are placed one over another in a direction perpendicular to an axial direction of the cylindrical casing 1, thereby forming the fluid flow path.

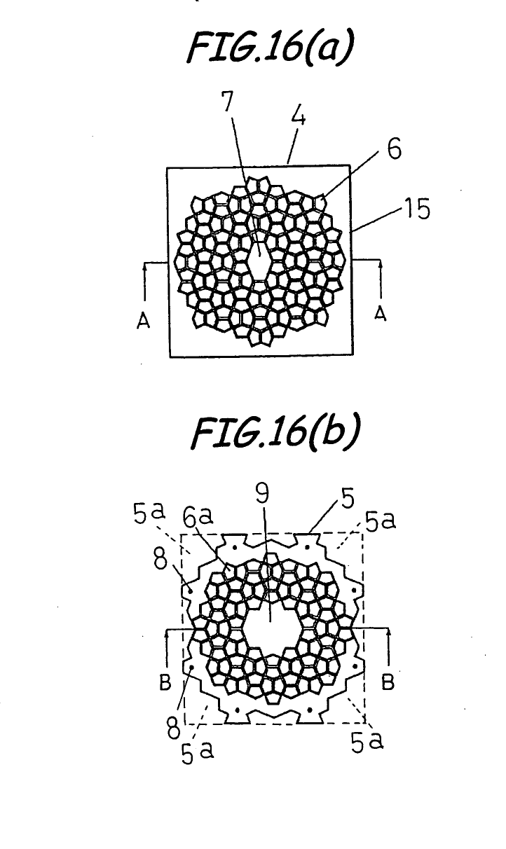

As shown in FIG. 16(a), each of the first plate members 4 has an outer circumferential shape that matches an inner circumferential shape of a hollow portion inside the cylindrical casing 1, and may be mounted inside the cylindrical casing I in such a way that it can make intimate contact with an interior of the hollow portion. Each first plate member 4 has a plurality of pentagonal (five-sided) cells 6 thereon that are arranged like a honeycomb. Each of the cells 6 has its front side open, and the cells 6 are configured to provide a central hole 7 though the configuration.

Each of the second plate members 5 has an outer circumferential shape such that the second plate member 5 can be mounted inside the hollow portion inside the cylindrical casing 1, by allowing a gap to be formed between an outer circumference of the second plate member 5 and an inner circumference of the hollow portion inside the cylindrical casing 1. Each second plate member 5 has a plurality of pentagonal (five-sided) cells 6a thereon that are arranged like a honeycomb. Each of the cells 6a has its front side open, and the cells 6a are configured to provide a central recess 9 thereon.

The apparatus shown in FIG. 15(a) includes the cylindrical casing 1 which may have an appearance of a rectangular shape or cylindrical shape (FIG. 22), and has flanges 10, 10 as shown in FIG. 15(b). Each of the flanges 10, 10 is located at each corresponding one of corners on a diagonal line across the cylindrical casing 1, extending outwardly. Those flanges 10, 10 are provided with bolts 11, through which the casing 1 can be assembled or disassembled easily.

In the embodiment shown in FIGS. 15(a) and 15(b), the cylindrical casing 1 has a hollow portion thereinside, whose inner circumference has a substantially square cross section. Similarly, the first and second plate members 4, 5 that are mounted in the casing I also have a substantially square cross section as shown in FIGS. 16(a) and 16(b).

The apparatus for mixing and/or crushing substances into fine particles has a cover 12 on each of the opposite ends thereof. Each of the covers 12 has a square cone shape, and may be mounted removably on each of the opposite ends. The cover 12 on an inlet side has the inlet opening 2 at a center, through which an object to be processed, such as being mixed or crushed, may be fed into the casing 1, whereas the cover 12 on an outlet side has the outlet opening 3 at a center, through which substances that have been processed, such as being mixed or crushed, may be discharged. The inlet opening 2 and outlet opening 3 may have any geometric shape.

In accordance with the present invention, component members that constitute the apparatus, such as the cylindrical casing 1, first and second plate members 4, 5 and others, may be made of any of metal or non-metal materials. Such materials may include carbons, composite metal materials such as combinations of carbon and copper, carbon and aluminum, carbon and magnesium, carbon and tungsten, carbon and titanium oxide, mineral materials such as ceramics, tourmaline and the like, and natural or synthetic resins.

As shown in FIG. 15(a), first and second structural members that may be installed inside the hollow portion of the cylindrical casing 1 for forming a fluid flow path includes first plate members 4 and second plate members 5, respectively, which are placed one over another in the direction perpendicular to the axial direction of the cylindrical casing 1. It should be noted that the first plate members 4 and second plate members 5 may be installed inside the hollow portion by placing those members one over another in a horizontal direction from the right toward left sides in FIG. 15(a).

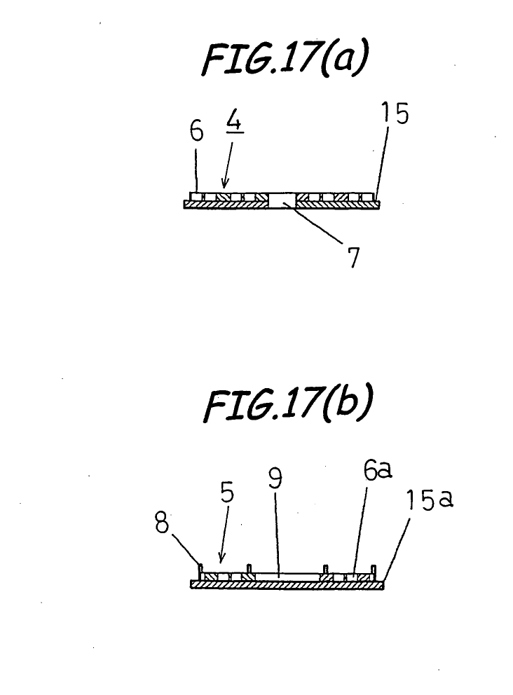

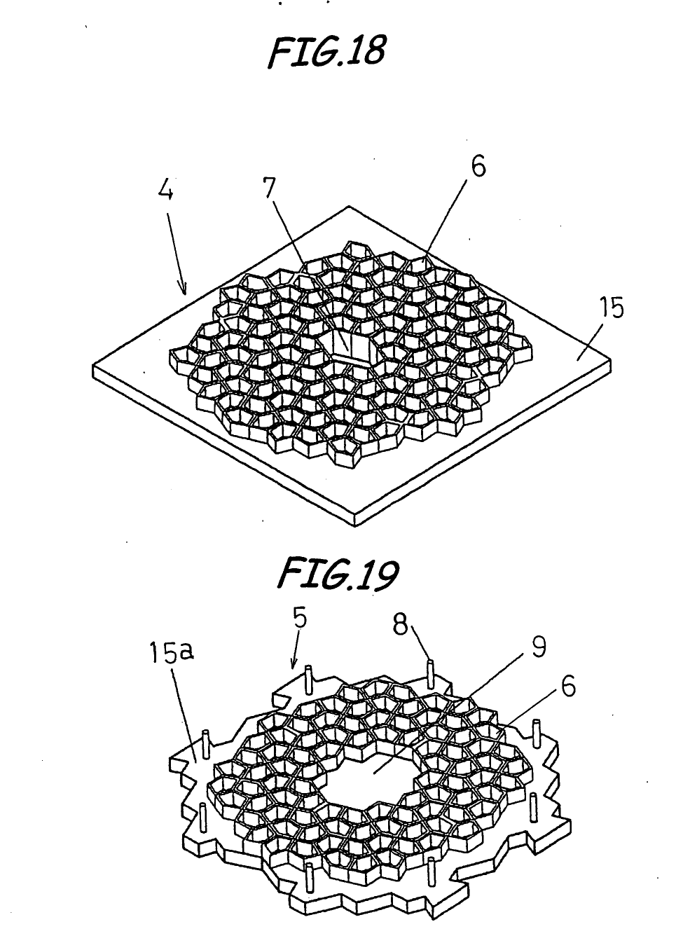

FIG. 16(a) is an enlarged plan view of the first plate member 4, FIG. 16(b) is an enlarged plan view of the second plate member 5, and FIG. 17(a) and FIG. 17(b) represent a cross section taken along line A-A in FIG. 16(a) and a cross section taken along line B-B in FIG. 16(b), respectively. FIG. 18 represents a perspective view of the first plate member 4, and FIG. 19 represents a perspective view of the second plate member 5.

It may be seen from FIG. 16(a) through FIG. 19 that the first plate member 4 includes a square base plate 15 that is slightly smaller than a section 14 located inside the cylindrical casing 1 where the plate member is mounted. The base plate 15 has a plurality of pentagonal cells 6 each having its front side open, and has a through hole 7 at its center that has a hexagonal shape having four longer sides and two shorter sides that is formed like a shape of a group of four cells 6. Outwardly of central hole 7, groups of four pentagonal cells 6 each having its front side open are arranged in a succeeding manner.

The second plate member 5 includes a square base plate 15a (FIG. 19) having four sides, each of which has cutout portions 5a at certain areas with two projecting sides. The base plate 15a has a recess 9 at its center that is formed like a shape of a group of fourteen (14) pentagonal cells 6a each having its front side open. Like the first plate member 4, outwardly of central recess 9, groups of fourteen (14) pentagonal cells 6a each having its front side open are arranged in a succeeding manner. The second plate member 5 has stability pins 8 (FIG. 19), each of which is provided on each of the projecting sides so that this can ensure stability of the first and second plate members when they are placed one over the other.

The second plate member 5 may include the base plate 15a that is formed to have cutouts on the four side corners 5a. The second plate member plate 5 may have any shape having an outer diameter that matches that of the first plate member 4. What is important is that there should be a space that allows a fluid to flow from one plate member to another adjacent plate member located downstream of the one plate member.

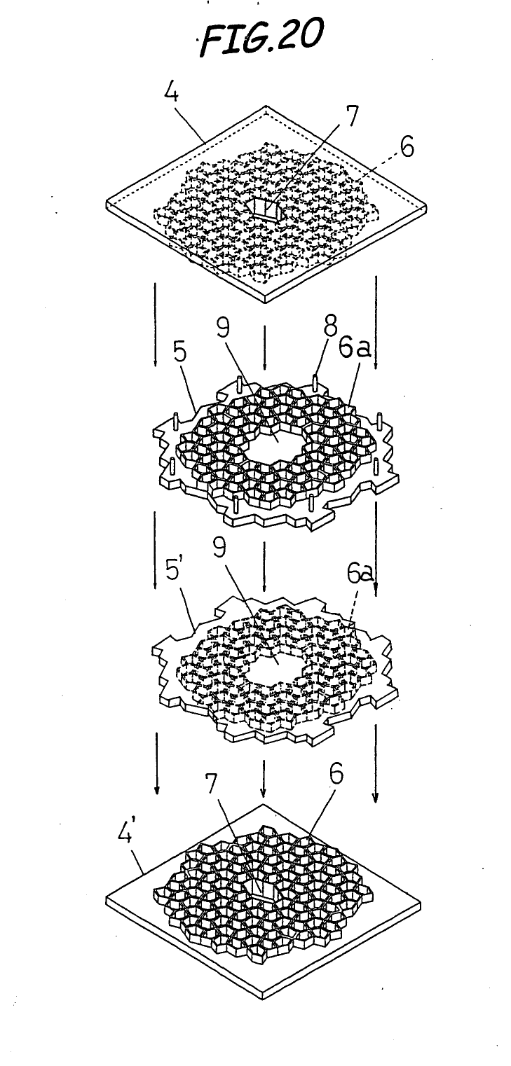

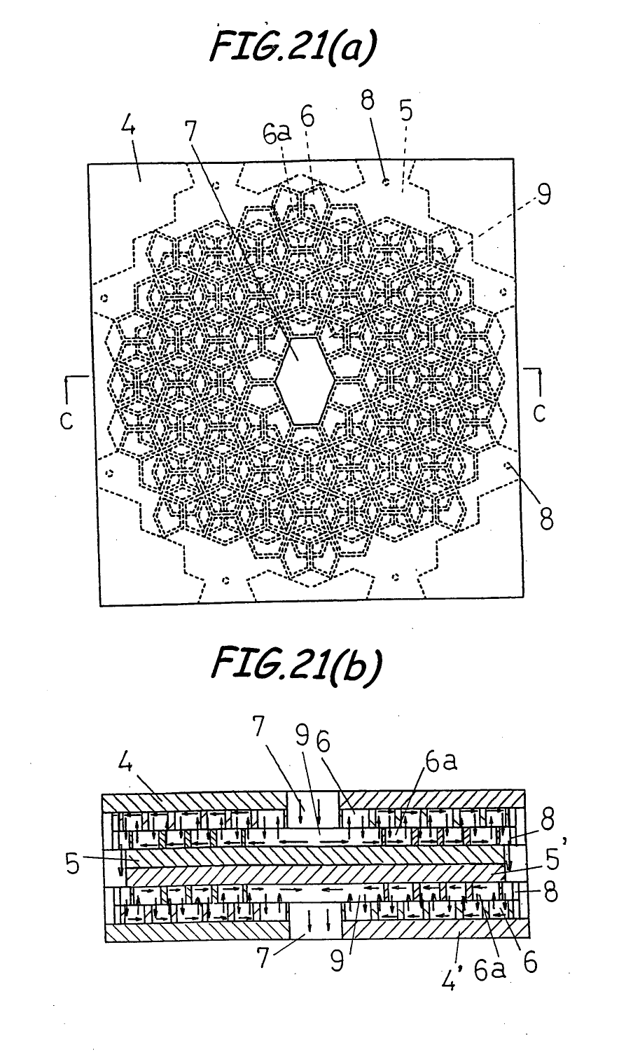

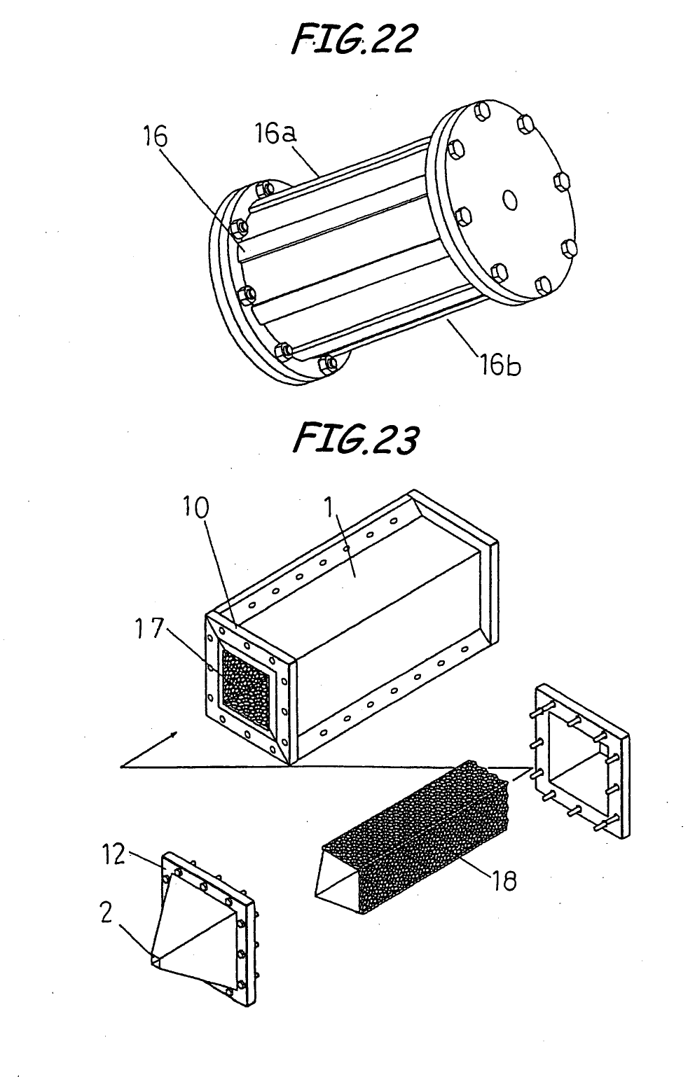

FIG. 20 represents a perspective view illustrating how the first plate member 4 and second plate member 5 are placed one over the other to form a single fluid conducting unit. FIG. 21(a) represents a plan view of the unit that includes the first and second plate members placed one over the other, and FIG. 21(b) represents a cross section taken along line C-C in FIG. 21(a).

The following describes how the first plate member 4 and second plate member 5, part of which is shown in FIG. 15(a), may be placed one over the other, by referring to FIG. 20.

Initially, the first plate member 4 and second plate member 5 may be placed one over the other so that the central hole 7 of the first plate member 4 can be aligned with the central recess 9 of the second plate member 5, with each of the pentagonal cells 6 having its front side open on the first plate member 4 being placed to face opposite each of the pentagonal cells 6a having its front side open on the second plate member 5 to communicate with each other through respective open front sides, and with each cell on the first plate member 4 being placed in alternate positions with regard to each corresponding cell on the second plate member 5. For example, the first and second plate members 4 and 5 may be placed by making intimate contact with each other so that each cell 6 on the first plate member 4 and each cell 6a on the second plate member 5 that is placed opposite each corresponding cell 6 on the first plate member 4 may be oriented reversely by turning one relative to the other by an angle of 90 degrees. Thus, when the first and second plate members are placed so that the cells 6 on the first plate member 4 may be placed to face opposite corresponding cells 6a on the second plate member 5, each of the cells on one plate member may communicate with at least one or more of the cells on the other plate member. Then, another second plate member 5′ may be placed back-to-back on the second plate member 5. Then the another plate member 5′ may be placed on another first plate member 4′ that is also placed back-to-back on a first plate member 4 so that the central hole 7 on the another first plate member 4′ can be aligned with the central recess 9 on the another second plate member 5′ and so that each of the pentagonal cells 6 having its front side open on the another first plate member 4′ is placed to face opposite each of the pentagonal cells 6 having its front side open on the another second plate member 5′ through respective open front sides, with each cell 6 on the another first plate member 4′ being placed in alternate positions with regard to each corresponding cell 6 on the another second plate member 5′. Thus, the another second plate member 5′ and another first plate member 4′ are placed one over the other so that each of the cells on one plate member may communicate with at least one or more of cells on the other plate member.

It may be appreciated that the fluid conducting unit may thus include the first plate member 4, second plate member 5, another second plate member 5′, and another first plate member 4′ that are placed one over another. FIG. 15(a) shows that several units are installed inside the cylindrical casing 1, but a single unit may also be installed.

It may be understood from the foregoing description that the first plate member 4 and second plate member 5 are placed one over the other such that these two plate members can contact each other intimately, with the pentagonal cells 6 on the first plate member 4 being placed opposite the pentagonal cells 6a on the second plate member 5, and with each of cells 6 being displaced relative to each of cells 6a by turning one relative to the other by an angle of 90 degrees. Thus, the cells 6 or 6a are arranged intimately like a honeycomb with no gap between any adjacent cells 6 or 6a as shown in FIG. 16(a) or 16(b), and a space on the front side of the cells 6 or 6a is divided into three parts as shown in FIG. 21(a).

When a fluid is introduced under applied pressure into the apparatus 13 so that the fluid can be mixed and/or crushed into fine particles, the fluid may first pass through the central through hole 7 of the first plate member 4, and may then hit the recess 9 on the second plate member 5, as shown in FIG. 21(b), flowing through a front space divided into three parts into upper and lower pentagonal cells 6, where the fluid hits the cells repeatedly, dispersing radially outwardly. The fluid that has dispersed or diffused up to an outer circumferential side goes to the base plate 15 in the first plate member 4 where it hits the base plate 15, flowing through the front space divided into the three parts into the upper and lower pentagonal cells 6, where the fluid hits the cells repeatedly, flowing toward a center. The above process, during which the fluid hits cells where its flow is reversed, is repeated throughout the cylindrical casing 1, and the fluid may be finally mixed and/or crushed into fine particles.

The components that constitute the apparatus 13 described above, such as the cylindrical casing 1, first plate member 4, second plate member 5 and the like, may be made of any of materials that may include carbons, metal-carbon combination materials such as carbon and copper, carbon and aluminum, carbon and magnesium, carbon and tungsten, and carbon and titanium oxide, and mineral materials such as ceramics, tourmaline and the like. The components that are made of any of the materials mentioned above may also provide an excellent catalyst action.

FIG. 22 is a perspective view illustrating an external shape of the cylindrical casing 1 as one component of the apparatus 13, which is formed like a cylindrical shape in this case.

In the embodiment shown in FIG. 22, the cylindrical casing 1 has its outer circumference divided into eight sections along the axial direction of the casing 1, and magnets 16 are disposed on the eight sections such that a magnet 16 on one section may provide N polarity 16a, for example, and a magnet 16 on a section opposite the one section may provide S polarity, for example. These magnets 16 may attract a fluid magnetically when it is introduced under applied pressure into the cylindrical casing 1 of the apparatus 13. Thus, molecules in the fluid may be subdivided more finely, and mixed and/or crushed into fine particles more efficiently under magnetic action of the magnets 16.

It should be noted that the cylindrical casing of the apparatus may have other external shapes. For example, the cylindrical casing 1 may include a flange that extends from a center along the horizontal direction, and may be divisible into two parts along the horizontal direction.

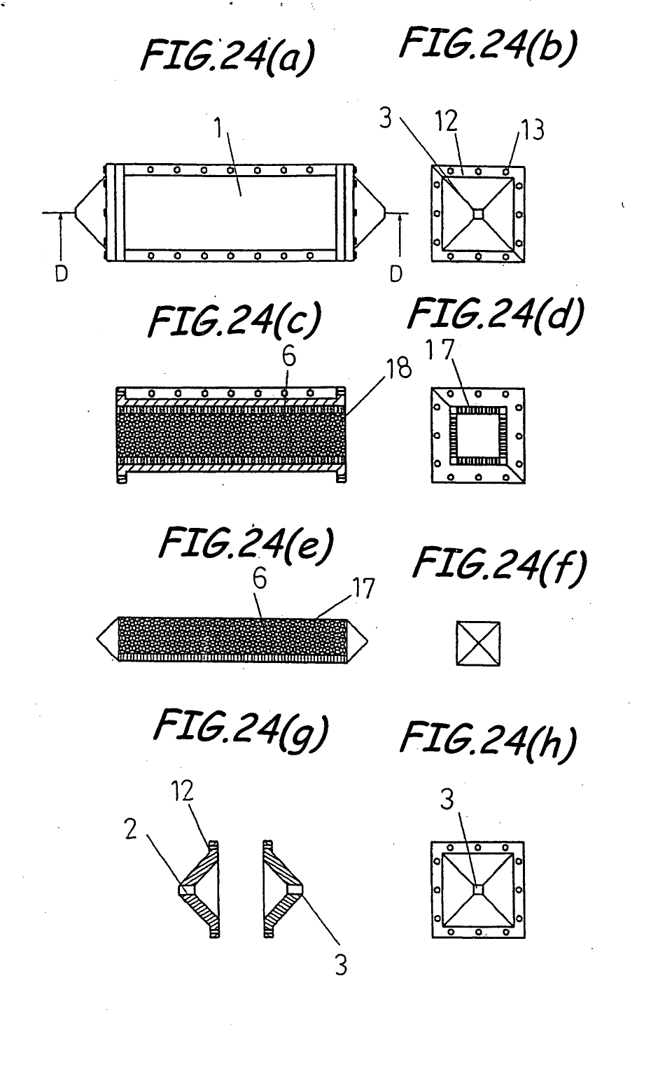

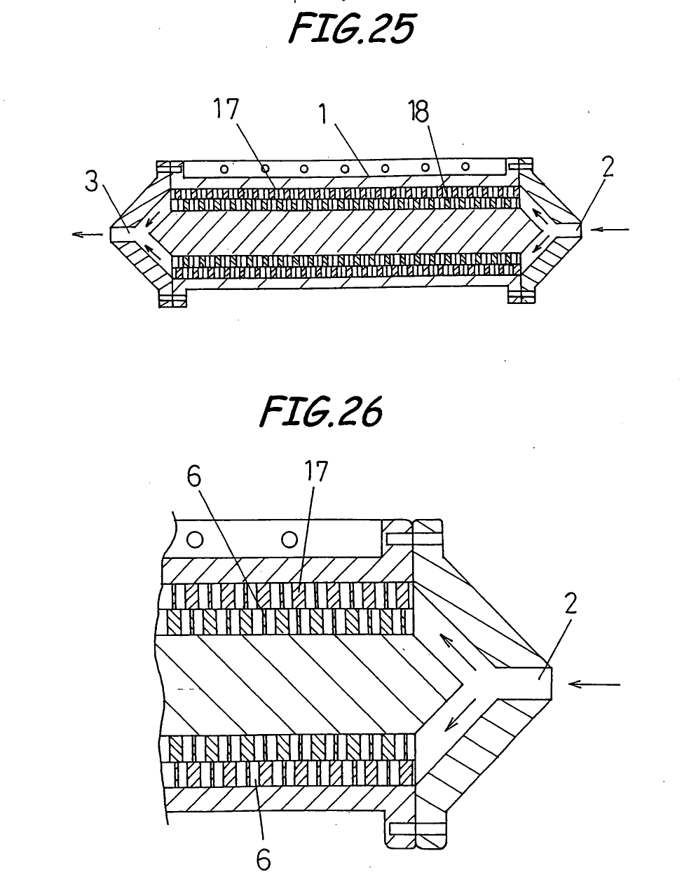

FIGS. 23 through 26 represent other possible embodiments. Specifically, FIG. 23 represents an exploded perspective view, FIGS. 24(a) through 24(h) represent an exploded view, and FIGS. 25 and 26 represent a sectional view and partly enlarged sectional view illustrating flow of fluid, respectively.

In these embodiments, the cylindrical casing 1 includes a plate member 17 having an inner circumferential wall formed like a square having four sides, and each of these four walls has a plurality of pentagonal cells 6 thereon, each having its front side open, that are arranged like a honeycomb. The cylindrical casing 1 further includes a structural member that may be fitted inside the cylindrical casing 1. The structural member is formed by a plate member 18 having an outer circumferential wall formed like a square having four sides, and each of these four walls has a plurality of pentagonal cells 6, each having its front side open, that are arranged like a honeycomb. With the plate member 18 being fitted inside the cylindrical casing 1, the pentagonal cells 6 on the plate member 17 may face opposite the pentagonal cells 6 on the plate member 18, with the former cells 6 being placed in alternate positions with regard to the latter cells. A fluid flow path may thus be formed through the open front sides of the cells facing opposite each other. The cylindrical casing further includes a flange on each of opposite ends, to which a cover 12 may be mounted.

Substances being processed, such as being mixed and/or being crushed into fine particles, take the form of a fluid, which may be introduced under applied pressure into the cylindrical casing 1 through inlet 2 on cover 12. Then, the fluid may flow into the space formed like a square pyramid in the cover 12, through which the fluid may go toward a location where the plate members 17, 18 are placed one over the other, with the open front sides of the pentagonal cells 6 on one plate member facing opposite the open front side of the cells 6 on the other plate member. Going through the fluid flow path formed by the pentagonal cells 6, the fluid may collide with a lateral side of each of the pentagonal cells 6, and may then reverse its flow. As the fluid flows though its flow path, it may hit a base, top and lateral sides of each pentagonal cell, and may then reverse its flow. Then, the fluid may be divided into several flows, which may collide with each other, and may then reverse respective flows when going further through each of the pentagonal cells 6. This process may be repeated through a total flow path until the fluid reaches outlet 3.

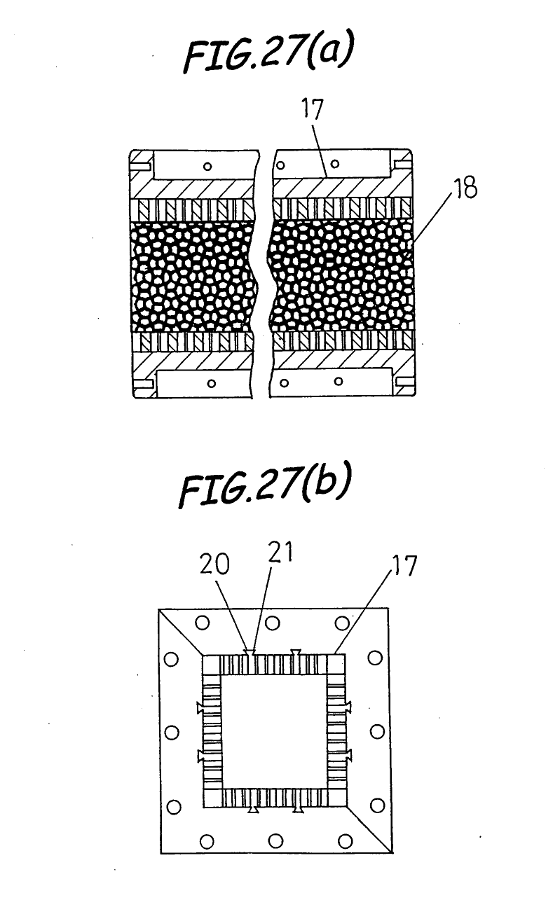

FIG. 27(a) through FIG. 29(b) represent other embodiments of the present invention, wherein a collection of continuous plate members 17 each having a plurality of pentagonal cells 6, each having its front side open, that are arranged like a honeycomb may be mounted on four inner sides of cylindrical casing 1, and a collection of continuous plate members 18 may be mounted on respective sides on base 19.

The cylindrical casing 1 and base 19 have a number of substantially triangular recesses 20 formed in appropriate locations on four faces thereof, respectively, and each plate member 17 and each plate member 18 have a number of substantially triangular projections 21 formed on sides opposite sides on which the pentagonal cells 6 are provided so that the projections 21 can engage corresponding recesses 20. These recesses 20 and projections 21 make it easy to replace the plate members 17 and plate members 18 that are made of different materials.

### Embodiment 2

In the preceding embodiment, each cell 6 having its front side open has a pentagonal shape. It should be appreciated that the cell may have any other shape other than pentagonal.

FIG. 30 represents other possible shapes that can be defined for the cells, each having its front side open, that are provided on the plate members.

As shown in FIG. 30, a right-angled isosceles triangle ΔABC having a right-angle vertex A is given, in which midpoints P and Q on each side are designated as absolute points. Then, an imaginary point S on a perpendicular from the vertex A to base B-C may be set on any location other than the vertex A and midpoint R on the base. Different segments that can be drawn to the imaginary point S, starting at midpoints P, Q on the sides may be turned clockwise about the midpoint P as an origin, and counter-clockwise about the midpoint Q as an origin. Then, the segments may intersect points S1, S2 on the base B-C. The shape that is surrounded by the points P-S-Q-S2-S1-P may be used as the shape for each of the cells having its front side open.

It may be appreciated that the imaginary point S may be set on any location on the perpendicular A-R, and a leg on the base may be formed by turning a segment connecting point S and the midpoints P and Q on the sides, with midpoints P and Q serving as origins, clockwise and counter-clockwise.

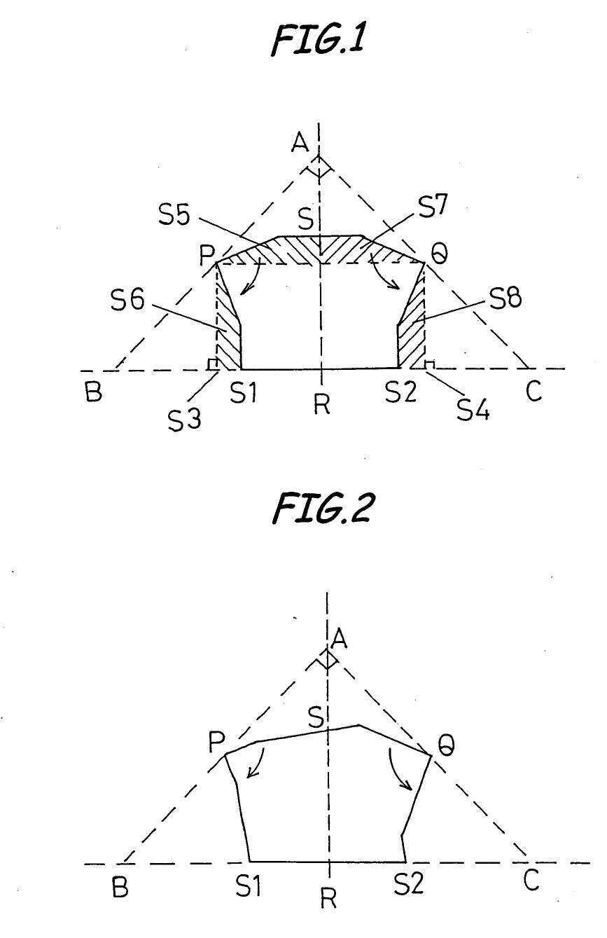

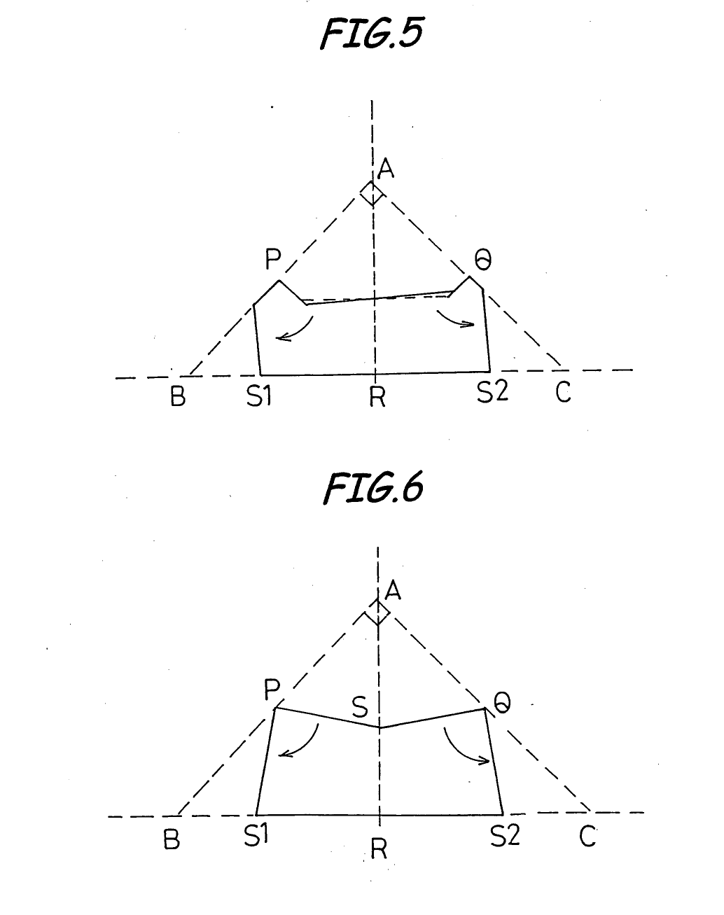

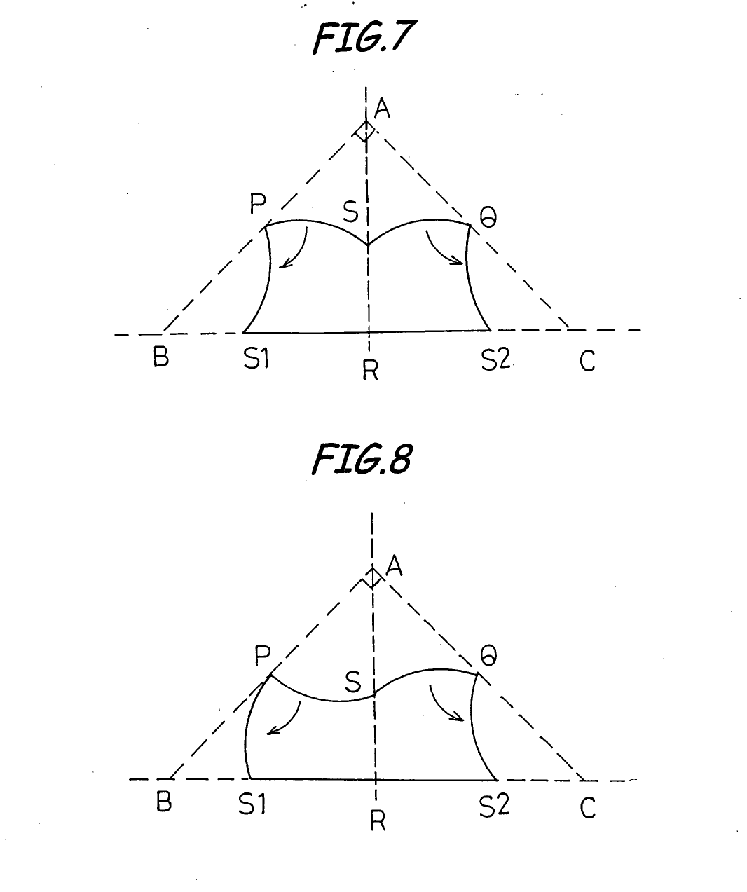

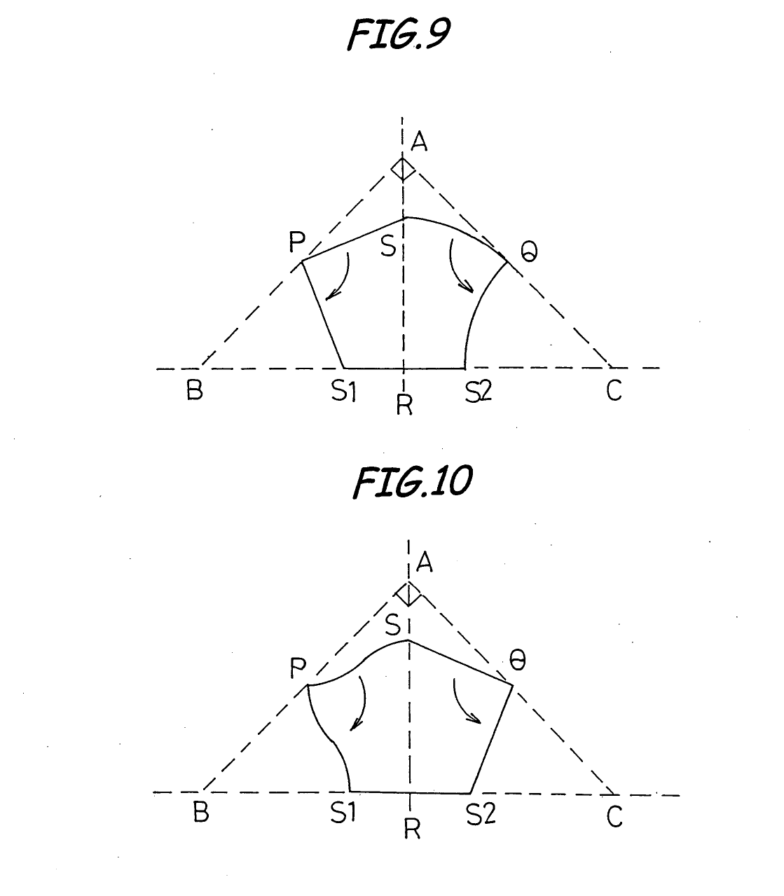

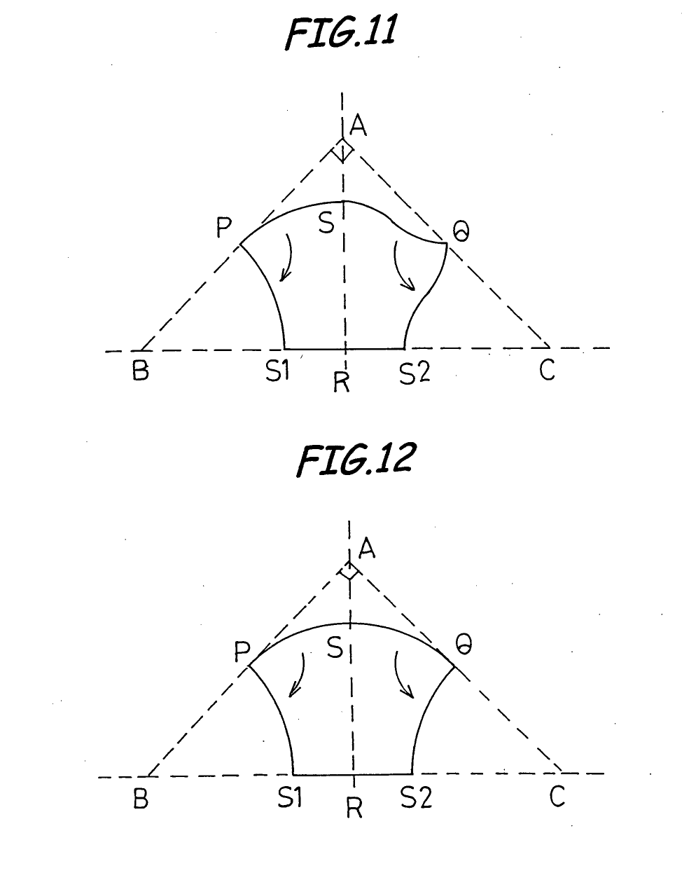

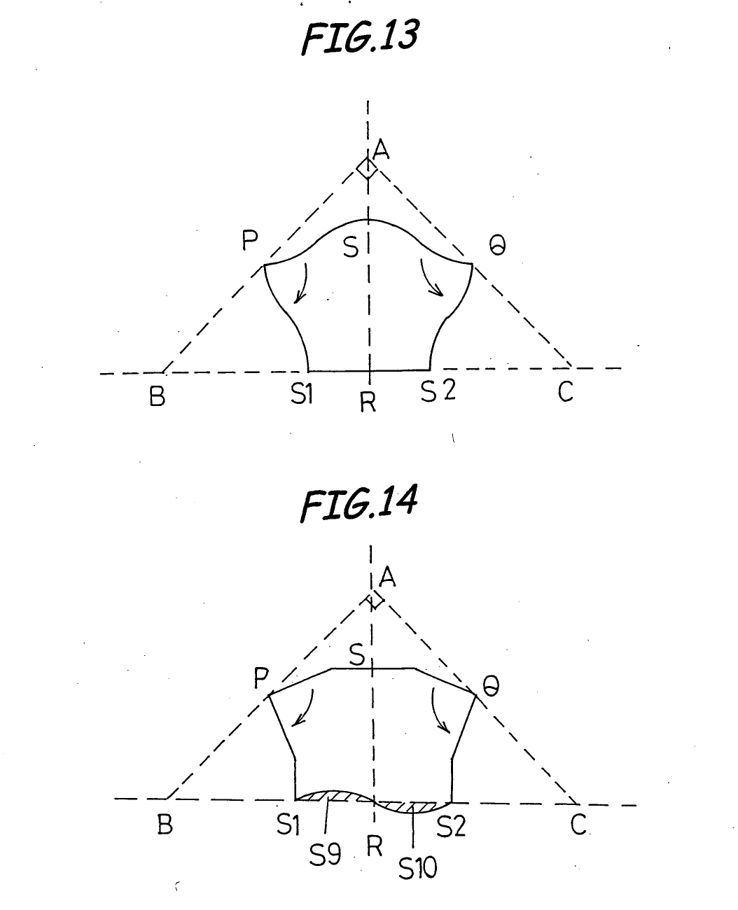

The cell may have other different shapes than the shape described above, as shown in FIGS. 1 through 14. Even when-the cells have any of the shapes shown, they can be placed on an entire plate member like a honeycomb, without producing any gaps between any two adjacent cells.

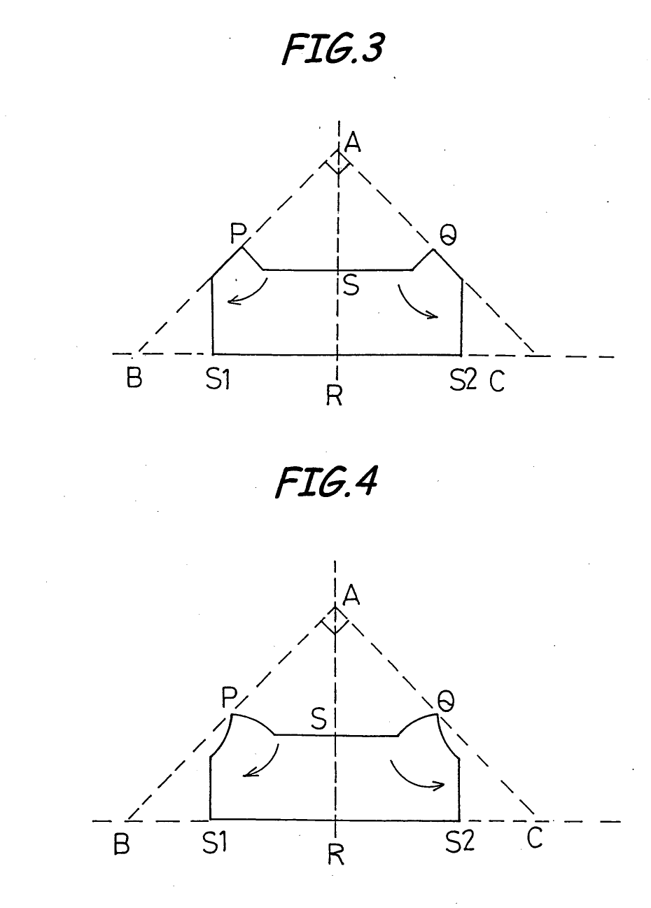

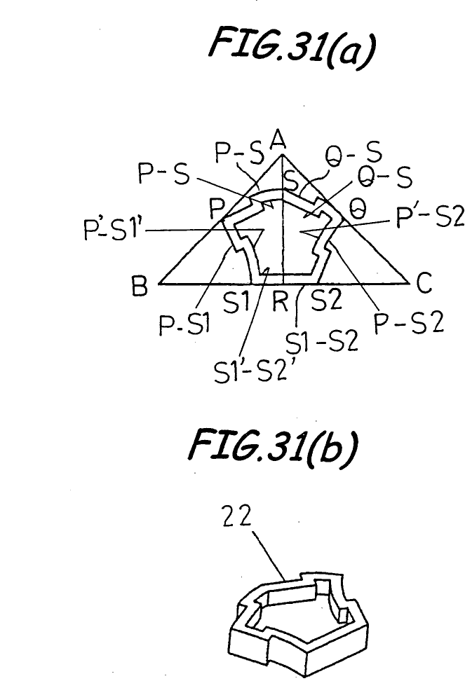

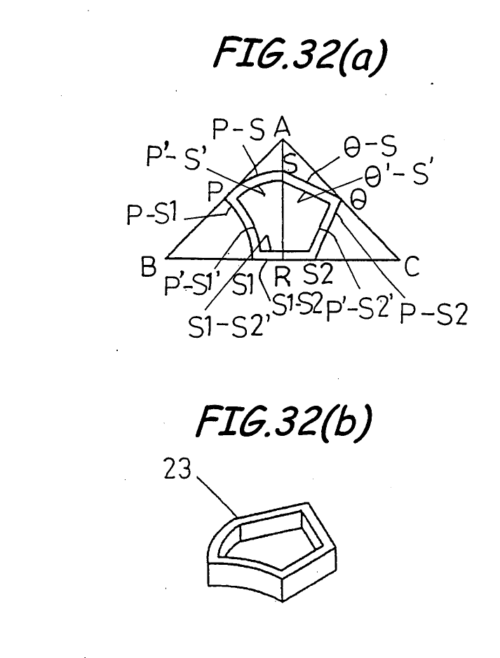

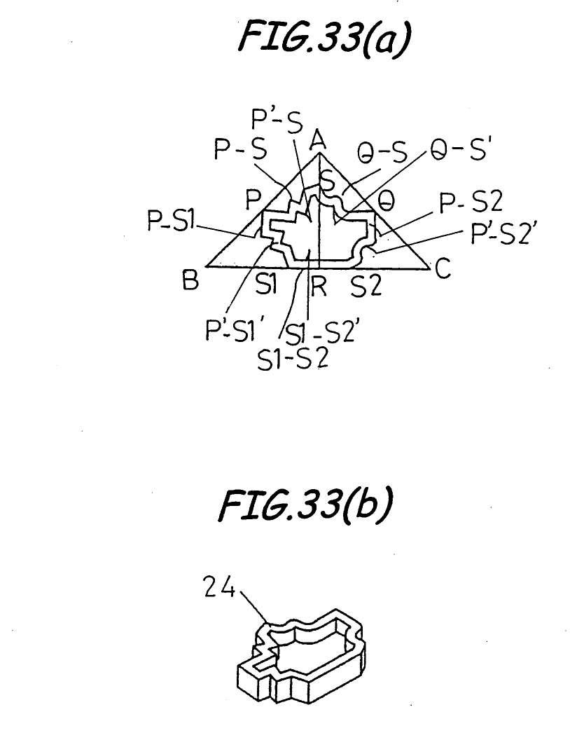

FIGS. 31(a) through 33(b) represent specific examples of cell shapes described by referring to FIG. 30, in which FIG. 31(b), FIG. 32(b), and FIG. 33(b) represent a perspective view of the cell formed in accordance with steps shown in FIG. 3 1(a), FIG. 32(a), and FIG. 33(a), respectively.

Specifically, it is shown in FIG. 31(a) that when an imaginary right-angled isosceles triangle ΔABC having right-angle vertex A, two sides A-B and A-C, and the base B-C is given, midpoints P, Q, R may be set on the sides A-B and A-C, and the base B-C, and point S may be set on any location other than points A, R on a line segment A-R connecting the vertex A and midpoint R. Segment P-S connecting midpoint P and midpoint S is a combination segment that includes an arc line from the midpoint on P-S toward point S, an arc line extending as straight line toward the midpoint and a straight line. The segment connecting midpoint Q and point S is a combination segment that includes a straight line from the midpoint on P-S toward point S, a straight line extending as an arc line toward midpoint Q, and an arc line. Then, the segment P-S may be turned clockwise about midpoint P as an origin, and the segment Q-S may be turned counter-clockwise about midpoint Q as an origin, and points of contact on the base B- C that result may be designated as S1 and S2. Connecting points P, S, Q, S2, S1, P may then produce an external shape that is asymmetrical. A portion that is sandwiched between the shape P-S-Q-S2-S1-P and the shape surrounded by segment P′-S′, segment Q′-S′, segment P′-S1′, segment S1′-S2′, and segment Q′-S2′ that is similar to but smaller than the above shape may be used as a wall of a cell 22.

It may be appreciated that base segment S1-S2 may be any other line type, not a straight line, such as the segment S1′-R that may be a sine line, and S2-R that may be a straight line. It should be understood, however, that it is desirable that the shape of the portion defined by P-S-Q-S2-S1-P should have an area that is equal to one half an area of the imaginary right-angled isosceles triangle.

In FIG. 32(a), when an imaginary right-angled isosceles triangle ΔABC having right-angle vertex A, two sides A-B and A-C, and base B-C is given, midpoints P, Q, R may be set on the sides A-B and A-C, and the base B-C, and point S may be set at any location other than points A, R on a segment A-R connecting the vertex A and midpoint R. Segment P-S connecting midpoint P and point S is designated as a convex arc line, and segment Q-S is designated as a straight line. Then, the segment P-S may be turned clockwise about the midpoint P as an origin, and segment Q-S may be turned counter-clockwise about the midpoint Q as an origin. Points of contact on the base B-C that may result are designated as S1 and S2. Connecting points P, S, Q, S2, S1, P may produce an external shape that is asymmetric. A portion that is sandwiched between the shape P-S-Q-S2-S1-P and a shape surrounded by segment P′-S′, segment Q′-S′, segment P′-S1′, segment S1′-S2′, and segment Q′-S2′ that is similar to but smaller than the above shape may be used as the wall of a cell 23, as shown in FIG. 32(b).

In FIG. 33(a), when an imaginary right-angled isosceles triangle ΔABC having right-angle vertex A, two sides A-B and A-C, and base B-C is given, midpoints P, Q, R may be set on the sides A-B and A-C, and the base B-C, and point S may be set at any location other than points A, R on a segment A-R connecting the vertex A and midpoint R. Segment P-S connecting midpoint P and point S is designated as a flexible broken line, and segment Q-S is designated as a corner line R of the flexible broken line. Then, the segment P-S may be turned clockwise about the midpoint P as an origin, and the segment Q-S may be turned counter-clockwise about the midpoint Q as an origin. Points of contact on the base B-C that may result are designated as S1 and S2. Connecting points P, S, Q, S2, S1, P may produce an external shape that is asymmetric. A portion that is sandwiched between the shape P-S-Q-S2-S1-P and a shape surrounded by segment P′-S′, segment Q′-S′, segment P′-S1′, segment S1′-S2′, and segment Q′-S2′ that is similar to but smaller than the above shape may be used as the wall of a cell 23, as shown in FIG. 33(b).

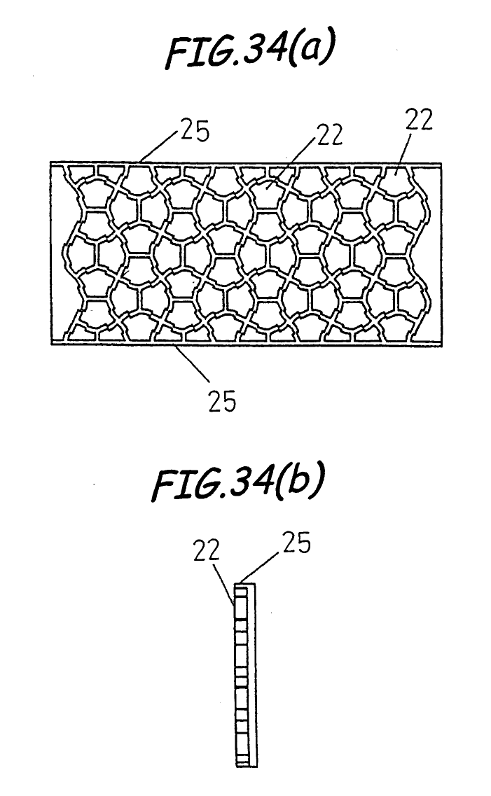

FIGS. 34(a) and 34(b) represent how the cells 22 having the shapes defined above are arranged like a honeycomb on a surface of a plate member. A top and bottom are formed by a wall 25 that is as high as the inner circumferential wall of the cylindrical casing 1 that forms part of the apparatus.

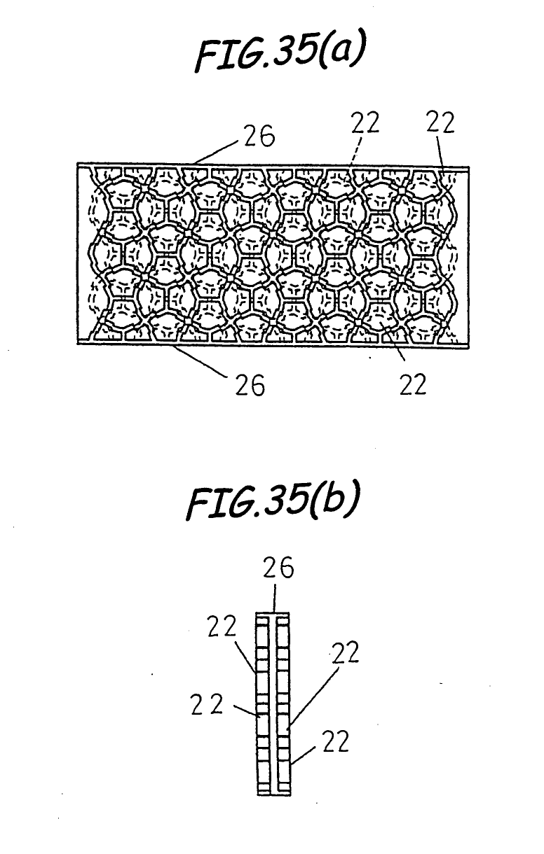

FIGS. 35(a) and 35(b) represent how the cells 22 having the shapes defined above are arranged like a honeycomb on front and rear sides of the plate member. When any two adjacent plate members are placed to adjoin each other, the cells 22 on the front side of one plate member and the cells on the rear side of the other plate member are arranged such that the cells on one plate member are placed in alternate positions with respect to the cells on the other plate member, with the cells on both plate members being displaced by an angle of 180 degrees. In this case, the top and bottom are also formed by a wall 25 that is as high as the inner circumferential wall of the cylindrical casing 1 that forms part of the apparatus according to the present invention.

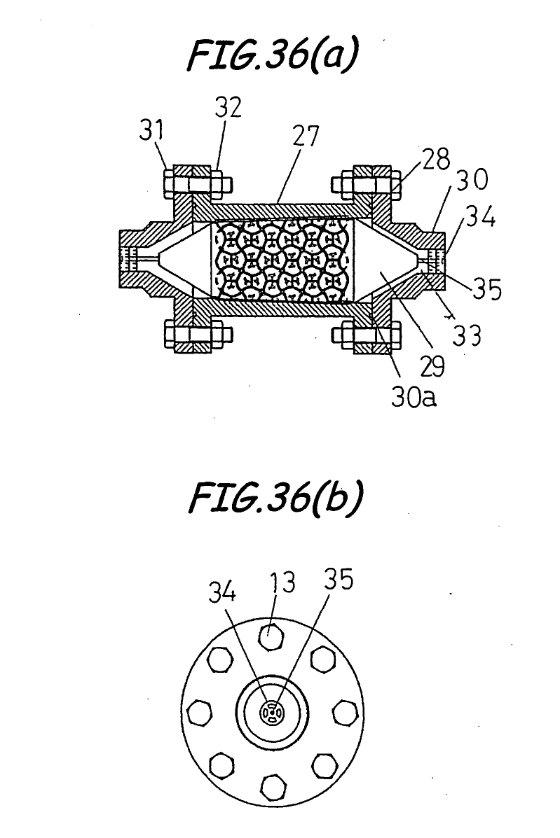

FIGS. 36(a) and 36(b) represent one specific example of the apparatus in accordance with the present invention, wherein the apparatus includes a plurality of plate members shown in FIGS. 35(a) and 35(b) that are placed one over another inside the cylindrical casing 1 in the axial direction of the cylindrical casing 1.

The cylindrical casing 1 has an appearance of a cylindrical casing 27 having a flange extending from each of opposite ends. The cylindrical casing 27 has an internal space 28 therein, which is formed like a frustoconical shape, and includes a plurality of plate members shown in FIGS. 35(a) and 35(b), which are arranged regularly on an entire inner circumferential wall of the internal space 28. A member 29 that may be fitted tightly into the internal space 28 has a conical shape.

Each of flanges on the opposite ends of the cylindrical casing 27 has an O-ring 30a that is provided as a seal for fluid. Cover 30 may be mounted on each of opposite ends of the cylindrical casing 27 by tightening bolt 31 and nuts 32, so that the cover 30 can keep the internal space 28 airtight. The cover 30 has an internal space 33 therein, which has a conical shape that is slightly larger than a conical shape on opposite ends of the member 29. Substances being processed, such as being mixed and/or being crushed, take the form of a fluid that may be fed into the cylindrical casing 27 through its inlet 34. Inside the inlet 34, there is a spiral fluid mechanism that is fastened by a bolt 35.

For the apparatus shown in FIG. 36(a), the inner space 28 has a frustoconical shape, and the member 29 fitted into the inner space 28 also has a frustoconical shape. When the fluid of substances to be mixed and/or crushed are fed into the cylindrical casing 1 through its inlet 34, the fluid may flow through paths through which each of the cells 22 on one of any two adjacent plate members placed one over the other in the axial direction of the cylindrical casing 1 can communicate with at least one or more cells on the other plate member opposite the one plate member, and can perform a mixing and/or crushing process during which fine particles can be generated or formed.

The apparatus shown in FIG. 36(a) may be used as a mixer, crusher or device for providing fine particles in spherical forms. It may also be used as a device for generating a critical fluid or ultra-critical fluid by causing substances being processed to react when pressure and temperature are placed under the ultra-critical condition above a gas-liquid critical point.

More specifically, the apparatus shown in FIG. 36(a) may be constructed by using structural members that are capable of resisting critical and ultra-critical temperature conditions under which substances are being mixed and/or crushed into fine particles. Thus, the apparatus shown in FIG. 36(a) may be used as a reactor vessel in which a process of mixing and/or crushing substances into fine particles can be performed under critical and ultra-critical processing conditions.

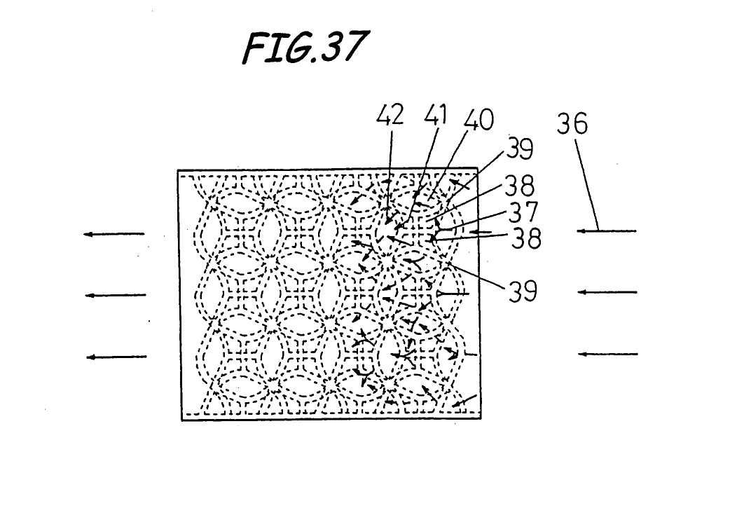

FIG. 37 is a diagram illustrating how substances being processed, such as being mixed and/or crushed, can flow through the fluid flow path within the cylindrical casing 27 in accordance with the embodiment shown in FIG. 36(a). There are two plate members that are placed adjacently to each other such that open front sides of the cells 22 on one plate member face opposite open front sides of the cells 22 on the other plate member, with the cells 22 on the one plate member being rotated through an angle of 180 degrees with regard to the cells 22 on the other plate member. These plate members may be installed within the cylindrical casing 27.

When a fluid of substances being processed is fed under applied pressure into the cylindrical casing 27 through its inlet 34, the fluid flows through a continuous sequence of divisional sections, flowing first into first divisional section 37 having an area and volume larger than other divisional sections, and then flowing into a next following divisional section 38. Fluid 36 flowing through the first divisional section 37 is blocked by walls 39 formed by midpoints P and P′ and midpoints Q and Q′ as described earlier and that overlap each other vertically, hitting against walls forming the cells 22 and going over the walls into the next following divisional section 38. The divisional section 38 has an area and volume that are smaller than the area and volume of the divisional section 37 by about 90%, and thus may include a number of divisions that is twice that of the divisional section 37. The fluid can flow faster when it goes through the divisional section 38.

Then, the fluid 36 goes to a next following divisional section 40 that has an area and volume that are increased by 2.15 as compared with the divisional section 38, where the fluid flows more slowly. Going further into a next following divisional section 41, the fluid can flow faster again, going into a next following divisional section 42 where the fluid flows more slowly.

It may be understood from the above description that the fluid 36 can flow through a continuous sequence of divisional sections having different areas and volumes, and when the fluid 36 passes through a smaller divisional section, it can flow at a faster rate, and can be placed under an embracing pressure that provides actions of increased compression and coagulation against the fluid. When the fluid 36 passes through a larger divisional section, on the contrary, it flows more slowly, and the embracing pressure is thus released from the fluid under which substances can be dissolved. By repeating a sequence of compression, coagulation and embracing pressure release as described above, high quality fine particles (such as fine particles having pure spherical forms) can be obtained.

(Examples of Test Cases)

The following describes results of testing that was performed under test conditions listed below, under which soybeans were crushed into fine particles by performing the method of the present invention using the apparatus of the present invention as shown in FIGS. 36(a) and 36(b).
Objects being tested: soybeans (containing dry fibers 40 to 300 μm long)
Applied pressure: 4.9 MPa (supplied by Pressure Pump)
Machine: apparatus of the invention shown in FIGS. 36(a) and 36(b)
   made of SUS316
   cylindrical type having length of 230 mm
   external diameter of 140 mm and internal diameter of 70 mm
   fluid flow path (a collection of plate members): 110 mm×50 mm made of SUS316
   a collection of plate members having a fluid flow path formed on front and rear sides (FIG. 35(a)): two sets
   a collection of plate members having a fluid flow path formed on a front side (FIG. 34(a)): two sets
      Electron microscope: power of 1000 (one scale division=1.538 micron)

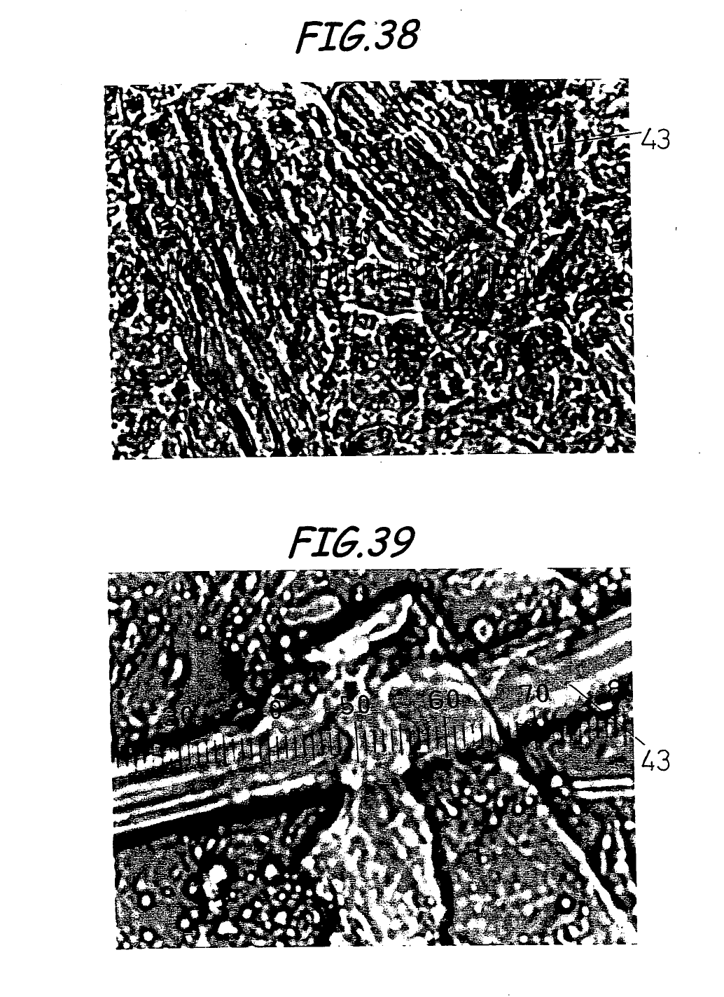

FIGS. 38 through 42 represent microscopic pictures showing how soybean fibers, which were obtained at time intervals of one, three and five minutes, can appear through this optical microscope, in which 10 liters of water was added to 2.0 kg of soybeans in their previously crushed forms, and a resulting mixture was fed under the applied pressure into the apparatus by use of the pressure pump, and the testing was performed at the above time intervals under the conditions listed above by circulating the soybeans through the apparatus.

In FIG. 38, reference numeral 43 refers to a state of the soybean fibers before being fed under the applied pressure.

FIG. 39 represents a partially enlarged microscopic picture that shows that soybeans 43 contain numerous fibers of different sizes.

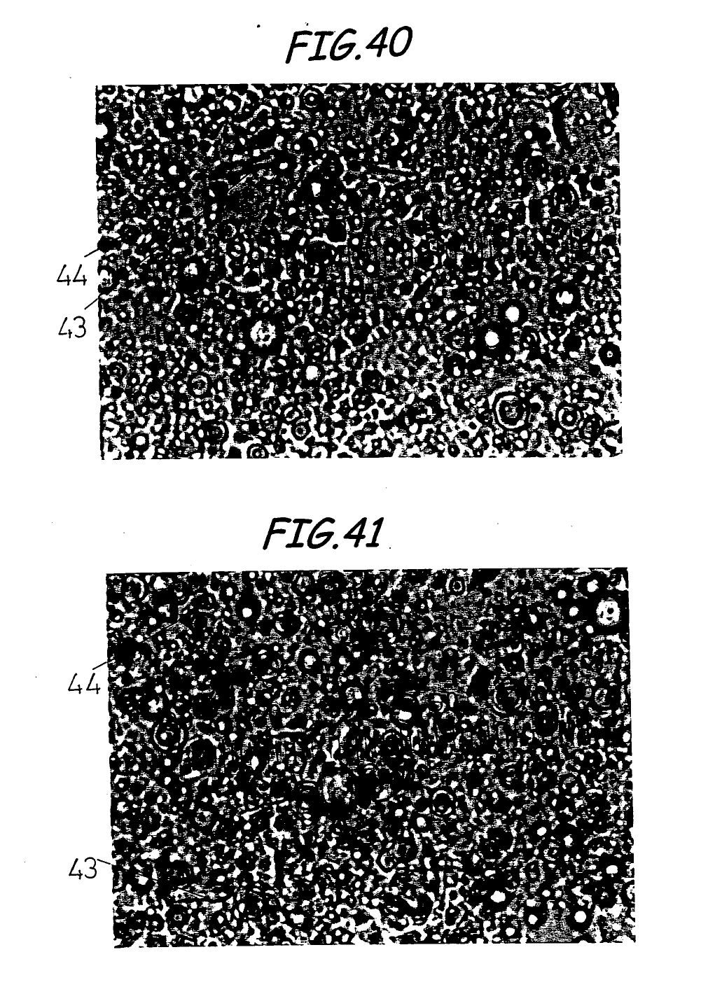

FIG. 40 represents a microscopic picture showing a state of the soybean fibers at an elapse of one minute after the soybeans were fed under the applied pressure. This picture shows that much of the soybean fibers have been crushed into fine particles, with some soybean fibers still remaining.

FIG. 41 represents a microscopic picture showing a state of the soybean fibers at an elapse of three minutes after the soybeans were fed under the applied pressure. This picture shows that the soybean fibers have mostly been crushed into fine particles, which are in nearly uniform forms.

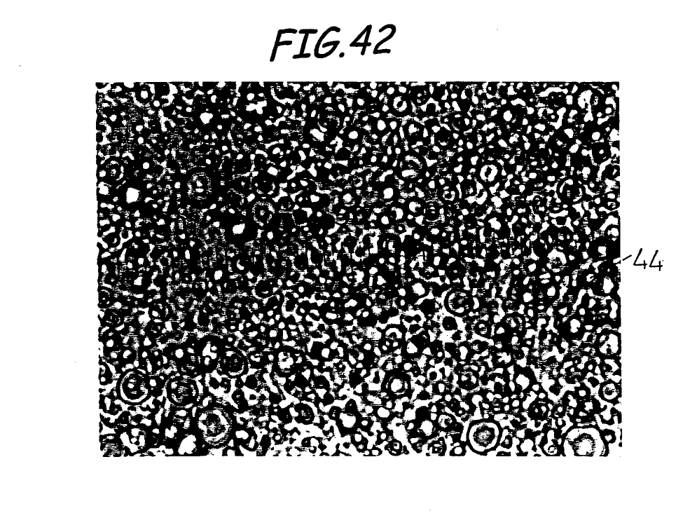

FIG. 42 represents a microscopic picture showing a state of the soybean fibers at an elapse of five minutes after the soybeans were fed under the applied pressure. This picture shows that all of the soybean fibers have been crushed into fine particles, with no soybean fibers remaining and with all fine particles 44 having spherical forms.

None of the methods and apparatuses that are known in this field permit a fluid of fibrous powders to be crushed into fine particles having nearly pure spherical forms, even by any of mixing, stirring, shearing, breaking and like operations. By using the methods and apparatuses of the present invention, it is possible to crush various types of fibrous materials into fine particles having nearly pure spherical forms.

### Embodiment 3

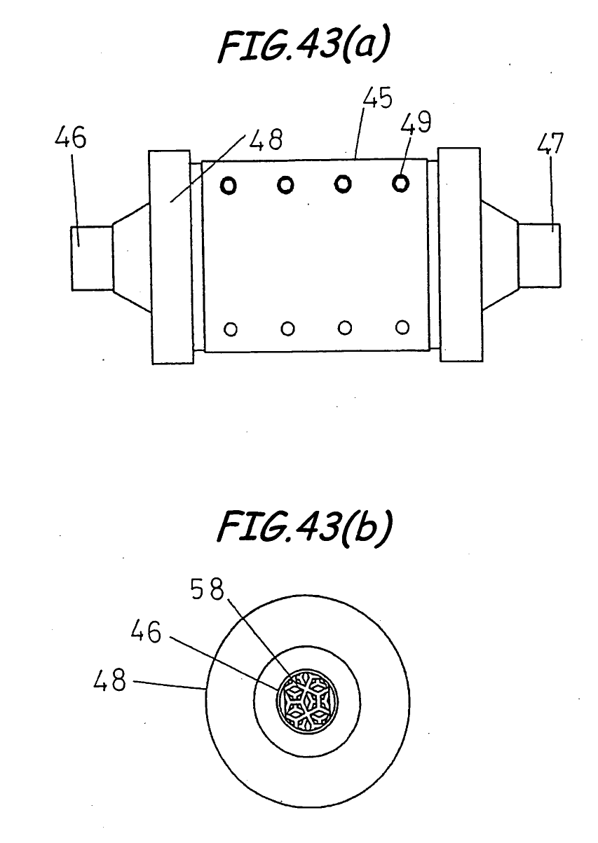

Referring now to FIG. 43, another embodiment of the apparatus for mixing and/or crushing substances into fine particles in accordance with the present invention is described. FIG. 43(a) is a front view of the apparatus including components constructed in accordance with the present invention, and FIG. 43(b) is a side elevational view.

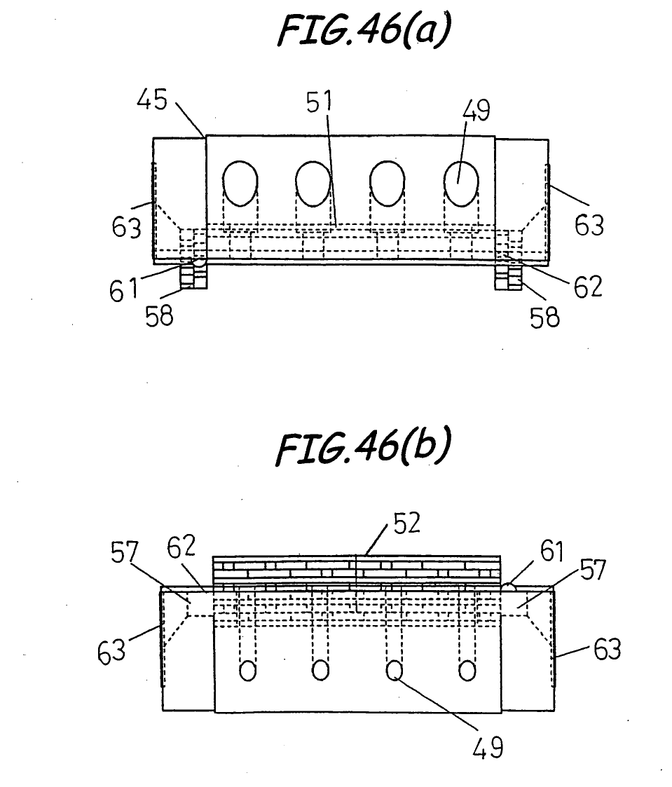

The apparatus comprises a cylindrical casing 45, and a cover 48 having an inlet 46 may be mounted to an inlet side of the casing 45, and a cover 48 having an outlet 47 may be mounted to an outlet side of the casing 45. The cylindrical casing 45 has a plurality of bolt insertion holes 49 provided at regular intervals on upper and lower portions. The cylindrical casing 45 has a divisible construction that permits the casing 45 to be divided into two parts in a horizontal axial direction as shown in FIGS. 45(a), 45(b) and FIGS. 46(a), 46(b).

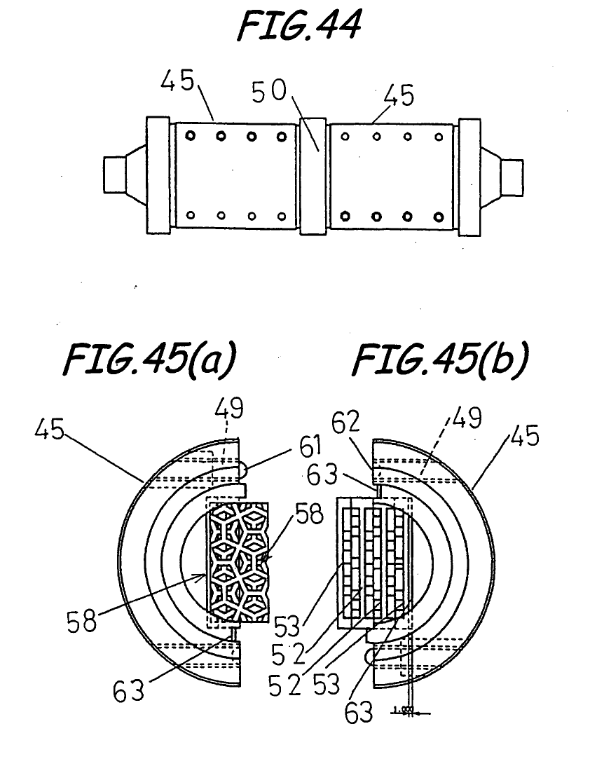

The apparatus may include two parts that can be connected together by connectors 50, as shown in FIG. 44.

FIGS. 45(a), 45(b), FIGS. 46(a), 46(b), and FIGS. 47(a), 47(b) show how the apparatus shown in FIGS. 43(a), 43(b) is divided into two parts in the horizontal axial direction.

FIGS. 45(a), 45(b) represent a side elevational view with some non-critical parts not being shown, in which FIG. 45(a) represents a side elevation of inlet side 46 or outlet side 47, and FIG. 45(b) represents a cross section in a central portion of the cylindrical casing 45 as viewed from a lateral side, with some non-critical parts not being shown.

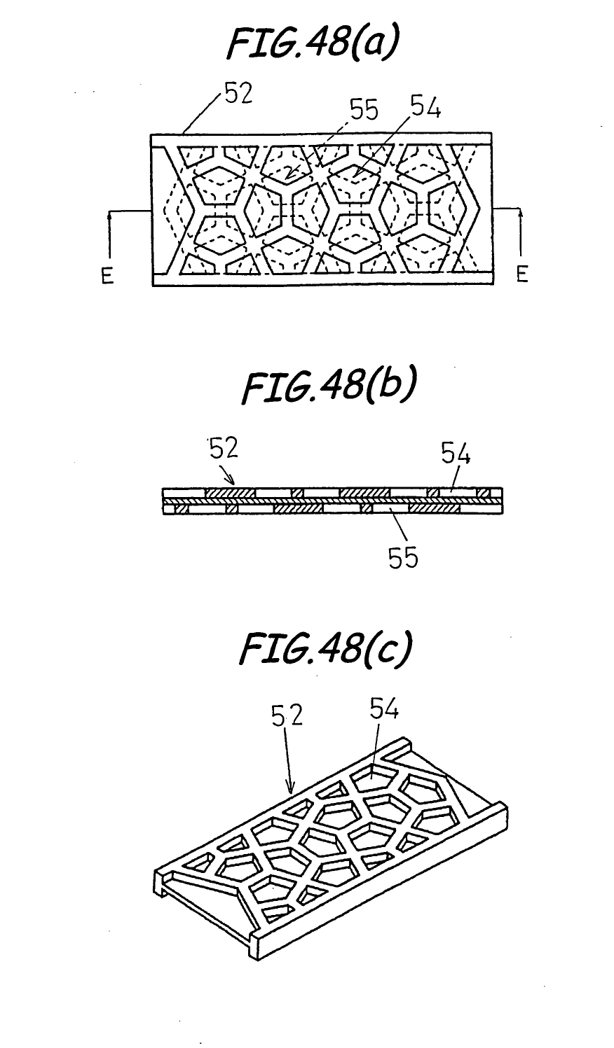

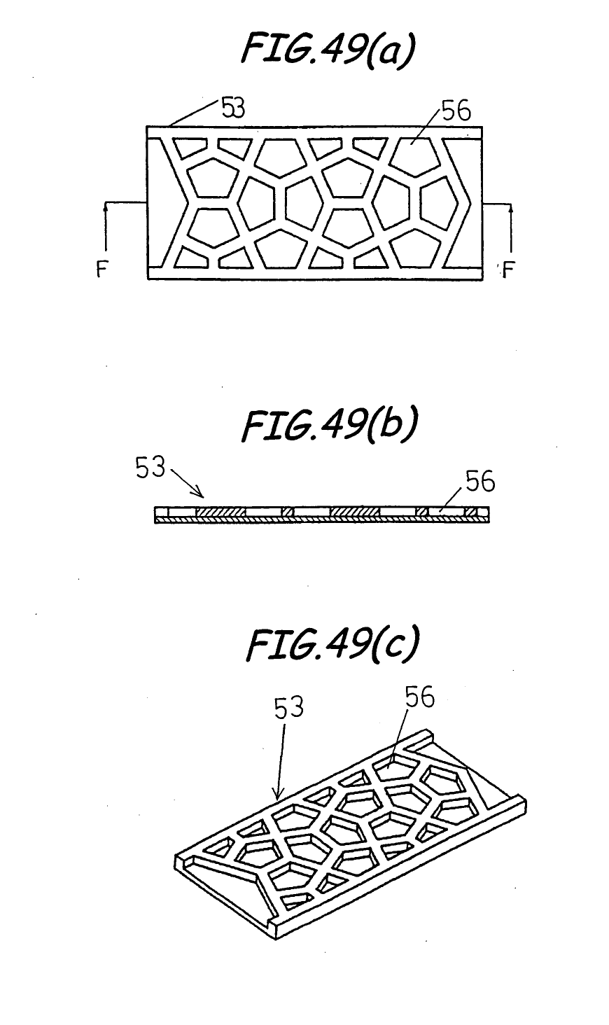

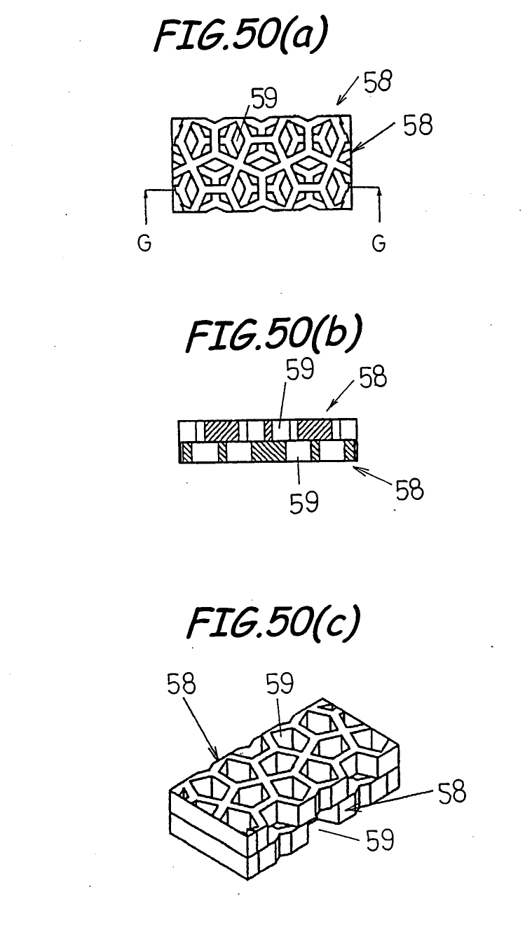

FIGS. 46(a), 46(b) correspond to a plan view, in which FIG. 46(a) illustrates how frame member 58 to be described in FIGS. 50(a) through 50(c) can be mounted and removed, and FIG. 46(b) illustrates the structural member 52 forming a fluid flow path to be described in FIG. 48(a) through FIG. 49(c) can be mounted and removed.

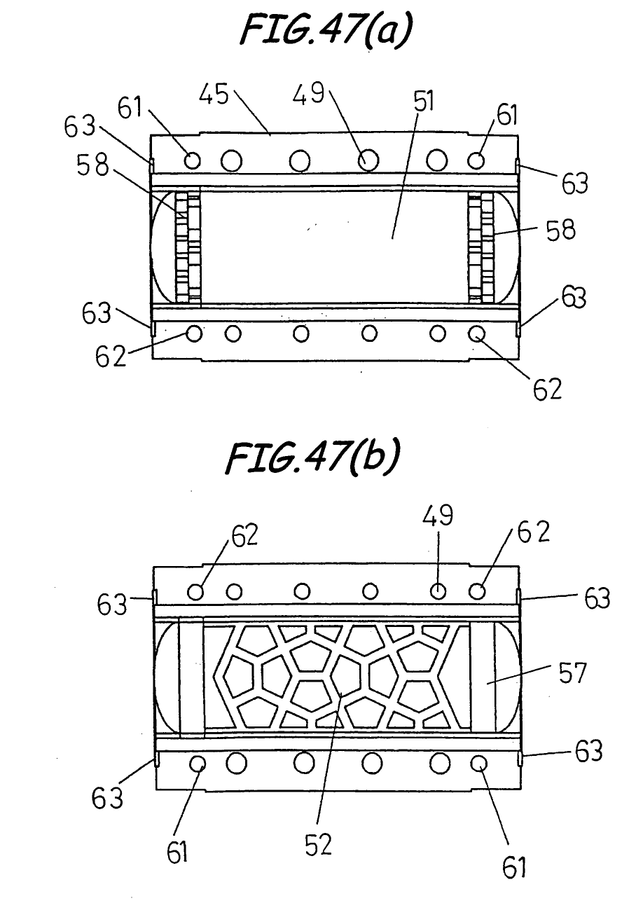

FIGS. 47(a), 47(b) correspond to a front view, in which FIG. 47(a) illustrates a portion of the fluid flow path that can be formed by the frame member 58 to be described in FIGS. 50(a) through 50(c), and FIG. 47(b) illustrates a portion of the structural member 52 to be described in FIG. 48(a) through FIG. 49(c).

The cylindrical casing 45 has a first recess 51 formed on an inner circumferential wall (FIG. 46(a)).

The first plate member 52 shown in FIGS. 48(a) through 48(c) and a second plate member 53 shown in FIG. 49(a) through 49(c) may be fitted in the first recess 51 and secured therein.

It may be seen from FIGS. 48(a), 48(b) that the first plate member 52 has a plurality of pentagonal cells 54, 55, each having its front side open, arranged on each of opposite sides thereof, respectively. In the embodiment now described, as shown in FIGS. 48(a), 48(b), the cells 54, each having its front side open, on an upper side of the first plate member 52 and the cells 55, each having its front side open, on a lower side of the first plate member 52 are all similar in shape, but the cells 54 on the upper side are arranged in alternate positions and in different positions by an angle of 180 degrees, with regard to the cells 55 on the lower side.

The second plate member 53 may have a plurality of pentagonal cells 56, each having its front side open, on at least one side thereof. In this embodiment, the cells 56 are arranged on an upper side of the second plate member 53, as shown in FIGS. 49(a), 49(b).

It may be seen from FIG. 45(b) that the first plate member 52 and second plate member 53 are arranged adjacently each other such that respective cells 54, 55, 56 on the first and second plate members can face opposite each other, with the respective cells 54, 55, 56 being placed in alternate positions with respect with each other and with each of the cells on one plate member communicating with at least one or more of the cells on another plate member.

As described above for the first plate member 52, the cells 54 on the upper side and the cells 55 on the lower side are placed in alternate positions with regard to each other, with each of the cells 54 being displaced by the angle of 180 degrees with regard to each corresponding one of the cells 55. As shown in FIG. 45(b), a fluid flow path that passes through the cells 54, 55 may thus be formed on an area where two adjacent first plate members 52, 52 are placed adjacently each other.

A fluid flow path may also be formed on an area where the first plate member 52 and the second plate member 53 are placed adjacently each other, as shown in FIG. 45(b). To this end, it is necessary that the pentagonal cells 56, each having its front side open, on the second plate member 53 may be placed in alternate positions, or displaced by a predetermined angle of 45 degrees, for example, with regard to any of the cells 54, 55 on the opposite sides of the first plate member 52 when they are placed to face opposite each other.

It may be appreciated that the embodiment shown in FIG. 45(b) may be varied such that two adjacent second plate members 53, 53 may be placed back-to-back (a lower side in FIG. 49(b)), and a fluid flow path may be formed by placing a first plate members 52 on each of opposite sides of these combined second plate members 53, 53 such that these first plate members 52, 52 can sandwich the second plate members therebetween.

This embodiment may further be varied to meet particular properties of substances being processed as well as particular processing requirements such as mixing and/or crushing into fine particles.

The cylindrical casing 45 also has a second recess 57 on the inner circumferential wall thereof (FIG. 46(b)).

Frame members 58, 58 that will be described in FIGS. 50(a) through 50(c) may be fitted in the second recess 57 and secured therein. The frame members 58 form a plurality of openings 59 arranged like a honeycomb and communicating with each other in the axial direction of the cylindrical casing 45. A plurality of frame members 58 are provided, and any two adjacent frame members 58 may be arranged one over the other in a direction perpendicular to the axial direction of the cylindrical casing 45, such that the openings 59 on one frame member can be placed in alternate positions with regard to corresponding openings 59 on the other frame member. In any of the embodiments shown in FIG. 45(a) and FIGS. 50(a) through 50(c), the openings 59 have a pentagonal shape.

FIGS. 48(a)-48(c) represent the first plate member 52, in which FIG. 48(a) corresponds to a front view, FIG. 48(b) corresponds to a sectional view along line E-E, and FIG. 48(c) corresponds to a perspective view. The first plate member 52 has a plurality of pentagonal cells 54, 55, each having its front side open, on the opposite sides thereof. As described, the cells 54 on the upper side are placed in alternate positions and are displaced by an angle of 180 degrees, with regard to the cells 55 on the lower side.

It should be understood that the present invention is not limited to the first plate member 52 described in this embodiment, but sizes of the cells 54, 55, each having its front side open, as described and shown, may be changed, and a number of cells 54, 55 may be increased or decreased, depending upon particular properties or a mixture ratio of substances being processed, such as mixing and/or crushing.

FIGS. 49(a)-49(c) represent the second plate member 53, in which FIG. 49(a) illustrates a front view, FIG. 49(b) illustrates a sectional view along line F-F, and FIG. 49(c) illustrates a perspective view.

The shape of the cells 56, each having its front side open, on one side of the second plate member 53 is similar to the shape of the cells 54, 55 on the first plate member 52, but as described earlier, the positions of the cells 56, each having its front side open, on the second plate member 53 are displaced by a predetermined angle, such as 45 degrees, with regard to positions of the corresponding cells 54, 55 on the first plate member 52, and the cells 56 are placed in alternate positions with regard to the corresponding cells 54, 55 when the former 56 is placed to face opposite the latter 54, 55.

Similarly, it should be understood that the present invention is not limited to the second plate member 53 described in this embodiment, but sizes of the cells 56, each having its front side open, as described and shown, may be changed, and a number of cells 56 may be increased or decreased, both depending upon particular properties or a mixture ratio of substances being processed, such as being mixed and/or crushed.

FIGS. 50(a)-50(c) represent how a plurality of frame members 58 that are placed one over another can be fitted easily in the second recess 57 and secured therein, in which FIG. 50(a) illustrates a front view, FIG. 50(b) illustrates a sectional view along line G-G, and FIG. 50(c) illustrates a perspective view.

The frame member 58 has a plurality of pentagonal openings 59, 59 extending through the frame member 58. Any two adjacent frame members 58, 58 are placed one over the other such that the openings 59 on one of the frame members facing opposite each other can be placed in alternate positions with regard to corresponding openings 59 on the other frame member.

The frame member 58 has an external shape that is similar to the shape of the second recess 57 on the inner circumferential wall of the cylindrical casing 45. This makes it easy to fit the frame member 58 in the recess 57 or to be removed from the recess 57.

It should be understood that sizes of the openings 59 that can be provided on the frame member may be changed, and a number of the openings 59 may be increased or decreased, both depending on particular properties and mixture ratios of substances being processed, such as mixed and/or crushed.

In FIGS. 45(a) through 47(b), a part denoted by 61 refers to a positioning projection, and a part denoted by 62 refers to a positioning recess. As described, the cylindrical casing 45 has a divisible construction that permits the cylindrical casing 45 to be divided into two parts in the axial direction thereof. Those two parts may be assembled by engaging the first plate member 52 and second plate member 53, and engaging the frame members 58, 58 with the second recess 51, followed by engaging the positioning projection 61 with the positioning recess 62 and then inserting bolts into the bolt insertion holes 49. Thus, this facilitates disassembling and reassembling of the cylindrical casing 45 for maintenance services.

In FIG. 45(a) through FIG. 47(b), a part denoted by 63 refers to a packing that provides a sealing function.

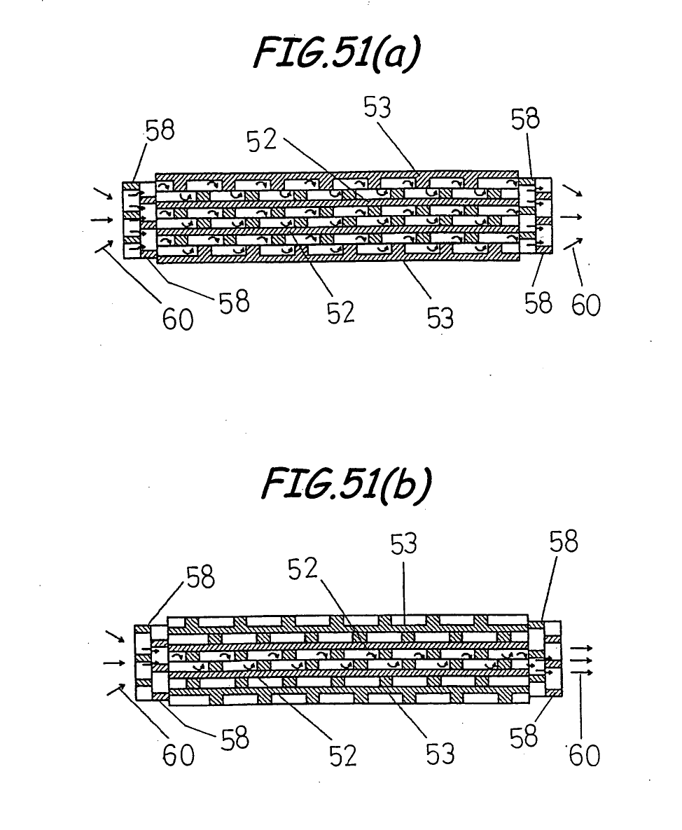

FIG. 51(a) is a schematic cross sectional diagram that illustrates how a fluid 60 of substances being processed, such as being mixed and/or crushed into fine particles, can flow through the fluid flow path formed by the frame members 58, 58 mounted inside the cylindrical casing 45 and through the fluid flow path formed by the first plate member 52 and second plate member 53, circulating through these fluid flow paths.

The fluid 60 may be delivered into the cylindrical casing 45 through the inlet 46 of the cover 48 by using any appropriate delivery device. Then, the fluid 60 may flow through the fluid flow path formed by the openings 59, 59 on the frame members 58, 58, producing some dispersing action.

Then, the fluid 60 flows through the fluid flow path formed by the first plate member 52 and second plate member 53, hitting against walls forming the cells 54, 55, 56 and repeating dispersing, swirling and reversing actions. During these repeated actions, the fluid may perform a process of mixing and/or crushing substances in the fluid into fine particles, flowing toward the outlet 47.

On a side of the outlet 47, the fluid 60 may flow through the fluid flow path formed by the openings 59, 59 on the frame members 58, 58 until the fluid reaches the outlet 47 on the cover 48 through which it exits.

FIG. 51(b) represents a schematic sectional diagram showing that placement of the first plate member 52 and second plate member 53 one over the other is reversed from the placement shown in FIG. 51(a), and how the fluid 60 can flow the fluid flow path in this case.

### Embodiment 4

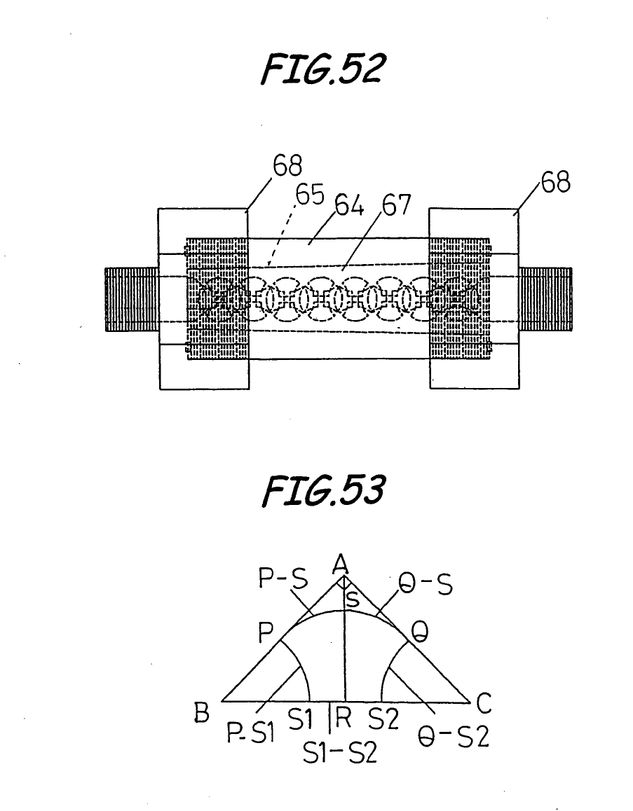

FIG. 52 through FIG. 55 illustrates another embodiment of the apparatus according to the present invention, wherein this apparatus includes cells that are formed so that they can form fluid flow paths in the manner described in FIG. 30 through FIG. 33(b), and includes plate members having such cells, each having its front side open.

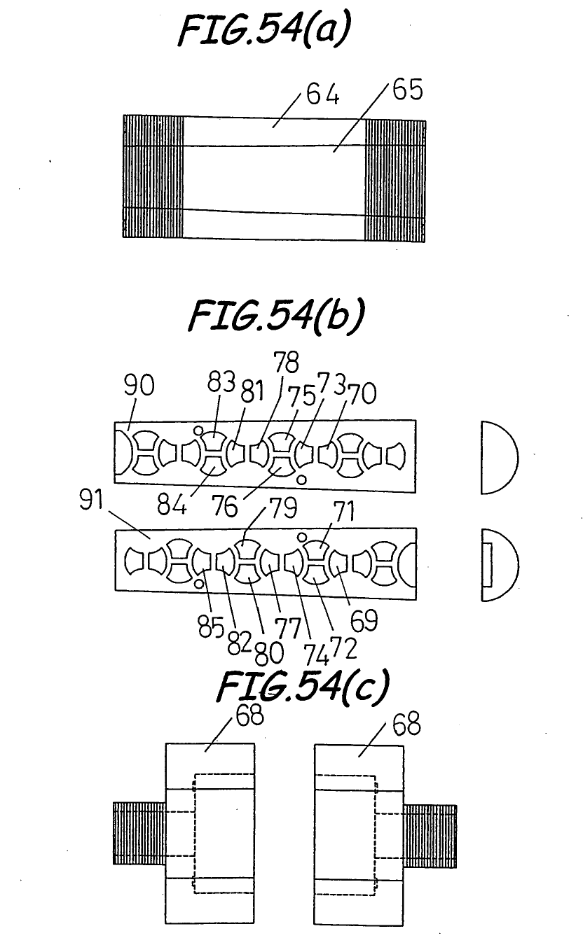

FIG. 52 represents a front perspective view with some non-critical parts not being shown, and FIG. 54(a) through FIG. 54(c) represent an exploded view of FIG. 52.

The apparatus shown in FIG. 52 includes a cylindrical casing 64 having an external cylindrical shape and having a hollow portion 65 therein. The hollow portion 65 may accept a fluid flow unit 67, which is so completely fitted inside the hollow portion 65 that it can prevent a short path from occurring.

A connecting screw 68, 68 may be removably mounted on each of opposite ends of the cylindrical casing 64 that may be used to connect the apparatus and any other external devices to the cylindrical casing 64. These connecting screws 68 may also serve to prevent the fluid flow unit 67 inside the hollow portion 65 from projecting out of the cylindrical casing 64.

The fluid flow unit 67 includes a plate member 90 having a plurality of cells 70, 72, 75, 76, 78, 81, 83, 84, each having its front side open, arranged on one side thereof, and a plate member 91 having a plurality of cells 69, 71, 72, 74, 77, 79, 80, 82, 85, each having its front side open, arranged on one side thereof, wherein the plate member 90 and plate member 91 are placed adjacently each other such that the cells on one plate member can face opposite corresponding cells on the other plate member, with the cells facing opposite each other being placed in alternate positions and with each of the cells on one plate member communicating with at least one or more of the cells on the other plate member. For example, the fluid flow unit 67 may be formed by placing the plate members 90 and 91 adjacently each other such that cell 69 having its front side open on the plate member 91 can communicate with cell 70 having its front side open on the plate member 90, whereby the cell 70 having its front side open on the plate member 90 can communicate with cells 71, 72 having its front side open on the plate member 91, and the cells 71, 72 having its front side open on the plate member 91 can communicate with cell 73 having its front side open on the plate member 90.

Thus, the fluid flow unit 67 may be formed by a sequence of plurality of cells 69 through 85 on one plate member, each of which communicates with at least one or more of the cells, each having its front side open, on the other plate member.

FIG. 53 illustrates a particular geometrical shape that may be used as a basis for defining a shape of each of the cells 69 through 85 on the plate members 90, 91 forming the fluid flow unit 67. Each of the cells 69-85 has a shape including a base defined as a straight line and other sides defined as arc curves.

A right-angled isosceles triangle ΔABC is given, where the triangle has a vertex A, a base B-C, and sides A-B and A-C. Then, arbitrary points P, Q may be set anywhere along the sides A-B and A-C, and an arbitrary point S may be set anywhere along a perpendicular between the vertex A and base B-C other than points A, R. Then, arc curves P-S and Q-S, connecting the arbitrary points P, Q on the sides and the arbitrary point S on the perpendicular, are turned about P, Q, respectively, such that P-S may be turned clockwise through an angle of 90 degrees, and Q-S may be turned counter-clockwise through an angle of 90 degrees. Then, points that are on the base B-C may be designated S1, S2. A shape that results is surrounded by arc curves P-S, Q-S, arc curves P-S1, Q-S2, and segment S1-S2 connecting points S1, S2.

Segments P-S and Q-S may have different types of line portions, such as straight, curved, sine curved, arced, broken and the like.

It is therefore important that an area of the shape surrounded by P-S-Q-S2-S1-P formed as described above should be equal to one half an area of the right-angled isosceles triangle ABC. In this way, the cells can be arranged on the plate members 90, 91 without any gaps being produced between any adjacent cells. As long as this condition is met, the arbitrary points P, Q, S may be placed on any locations on the sides A-B, A-C and on the perpendicular A-R.

It should be noted that a segment connecting point S1 and point R on the base may be straight, and a segment connecting point S2 and point R on the base may be curved.

It should also be noted that the fluid flow unit 67 may have a columnar shape that includes conical portions on opposite ends thereof that may be separated from each other, and may be recombined into one unit by use of guide pins provided at appropriate locations.

In addition, it is desirable that the cells 69 through 85 on the plate members 90, 91 forming the fluid flow unit 67 should be placed in respective positions that are displaced relative to each other by rotating one plate member relative to the other plate member through an angle of 180 degrees, as shown in upper and lower portions of FIG. 54(b), when the plate members 90, 91 are placed adjacently each other such that the open front sides of the cells on one plate member can face opposite those of the cells on the other plate member. In this way, the cells 70, 73, 75, 76, 78, 81, 83, 84 on one plate member 90, and the cells 69, 71, 72, 77, 79, 80, 82, 85 on the other plate member 91 that face opposite the cells on the one plate member 90 can divide a respective front space into a number of space portions, and each of the cells on each of the plate members can communicate with at least one or more of the cells on the other plate member.

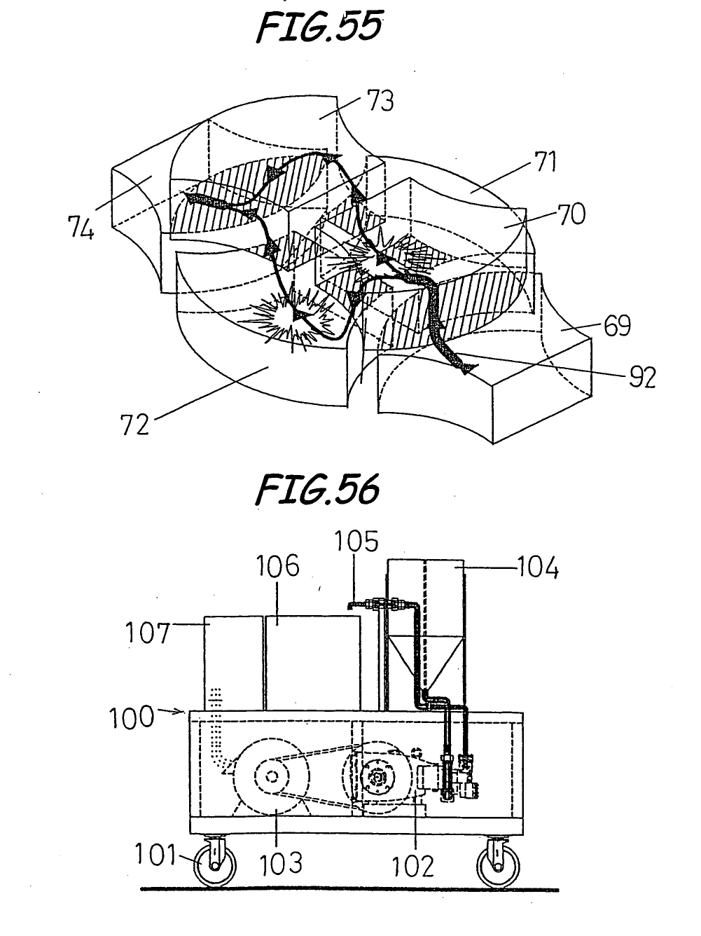

FIG. 55 is a schematic diagram illustrating how fluid 92 of substances being processed, such as being mixed and/or crushed into fine particles, can flow through the fluid flow unit 67. By referring to FIG. 55, flow of the fluid is explained below.

The fluid 92 first enters a cell 69 on lower plate member 91, and then enters a cell 70 on opposite plate member 90. Then, the flow 92 is divided into two flows that flow into cells 71, 72 on opposite plate member 91. These two flows rejoin each other in a single flow that enters a cell 73 on the opposite plate member 90. The fluid that exits the cell 73 then enters a cell 74 on the opposite plate member 91, where the flow is divided into two flows that enter cells 75, 76 on the opposite plate member 90. Then, these two flows rejoin each other into a single flow that enters a cell 77 on the opposite plate member 91.

While the fluid 92 is flowing through the cells as described above, the fluid 92 goes through a continuous process of dispersing, concentrating, rejoining, compressing by pressure, and releasing from pressure, and this process is repeated until the substances can finally be mixed and/or crushed into ultrafine particles or molecules.

### Embodiment 5

FIG. 56 is a general arrangement diagram illustrating one example of the apparatus described above in accordance with embodiments 1 through 4, in which the apparatus may be used to crush soybeans into ultrafine particles. The apparatus generally shown by 100 is equipped with casters 101 that permit the apparatus to travel. The apparatus 100 contains a drive motor 103 on a bottom for driving a pressure pump 102, and includes an inverter 107. In addition, a hopper 104 is mounted on top of the apparatus, through which soybeans may be delivered into the apparatus, and a return vessel 106; is provided near an outlet 105 of the apparatus 100, into which the soybeans just processed, such as crushed into ultrafine particles, may be collected.

An operation is now described. When a fluid of soybeans is placed into the hopper 104, the fluid goes through delivery piping where the fluid is placed under appropriate pressure supplied by the pressure pump 102, going into the apparatus 100 through an inlet (not shown). Then, the fluid flows through the fluid flow paths in the fluid flow unit 57 described in FIG. 52, and FIGS. 54(a), 54(b) and in the manner described in FIG. 55. When entering the cells 69 through 85 under the applied pressure, the soybeans may be compressed strongly by this strong pressure, followed by being released immediately from such compression. Thus, the soybeans may continue to be broken into ultrafine particles by internal and external pressures released by exploding themselves, and the ultrafine particles may be collected into the return vessel 106 through the outlet 105. This process of the soybeans continuing to be broken into ultra-fine particles, by the internal and external pressures released, by exploding themselves when the soybeans are flowing through the fluid flow paths as described in FIG. 55 may be explained by the so-called “dissipation theory”.

### Embodiment 6

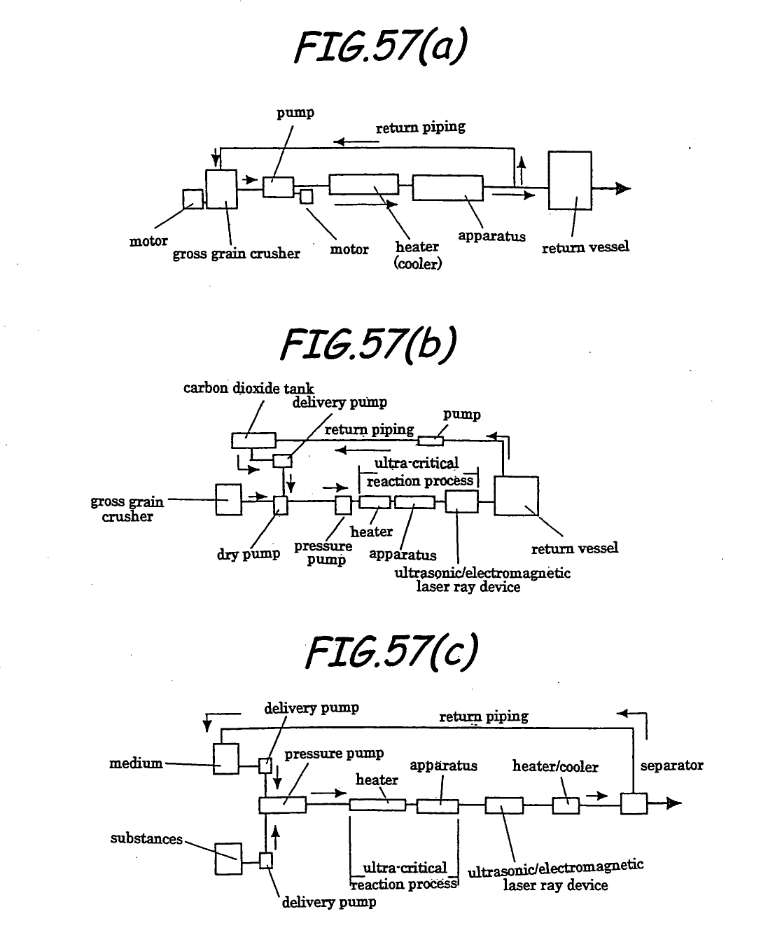

FIG. 57(a) through FIG. 57(c) are block diagrams illustrating a process of a method according to several embodiments of the present invention that may be implemented on the apparatus described above in embodiments 1 through 4.

FIG. 57(a) is a schematic block diagram illustrating one example of a method that includes steps of crushing wet-type substances into fine particles that may be implemented with the apparatus of the invention. This process is now described below by referring to FIG. 57(a).

Raw substances being processed, such as being mixed and/or crushed into fine particles, may first be supplied into a gross particle crusher where the raw substances are crushed into grossly crushed particles that may be delivered into a heater by a delivery pump, from which the grossly crushed particles may be delivered into fluid flow paths in the apparatus of the present invention. Passing through the fluid flow paths, the particles may be crushed into finer particles having desired particle sizes and then be collected in a storage vessel. A filter that is disposed between the apparatus and the storage vessel may filter the particles, and those particles that have not passed through the filter may be returned to the gross particle crusher where they may be crushed into finer particles again as described above and then collected in the storage vessel. The particles in the storage vessel may be delivered to any following step (finishing line).

FIG. 57(b) is a schematic block diagram illustrating one example of a method that includes steps of crushing substances into fine particles by causing the substances to react under continuous ultra-critical processing conditions using carbon dioxide that may be implemented by the apparatus of the present invention in combination with ultrasonic waves/electromagnetic wave/laser ray illuminating devices. This process is now described below by referring to FIG. 57(b).

Raw substances that have previously been crushed into gross particles may be mixed with any extracted solvent such as carbon dioxide through a delivery pump and dry-type pump, and this resulting mixture may be brought to appropriate pressure and temperature levels by use of a pressure pump and heater that would cause the mixture to be placed under continuous ultra-critical conditions. Then, the mixture may be delivered under this applied pressure into the cylindrical casing in the apparatus. While flowing through fluid flow paths, the mixture may be placed under the continuous ultra-critical conditions, under which the mixture may be crushed into ultrafine particles. The substances thus crushed into the ultrafine particles may then be exposed to ultrasonic waves, electromagnetic waves, laser rays, and the like so that they can react chemically or can be dissolved.

A final product thus obtained may be collected into a return vessel, and an extracted solvent in its liquefied state may be delivered to a pressure control valve (not shown), through which the solvent may be gasified for recycling.

FIG. 57(c) is a schematic block diagram illustrating one example of a method that includes steps of crushing substances into fine particles by causing the substances to react under continuous ultra-critical processing conditions using some types of solvents that may be implemented by the apparatus of the present invention in combination with ultrasonic waves/electromagnetic wave/laser ray illuminating devices. This process is now described below by referring to FIG. 57(c).

Any extracted solvent in its liquid state and the substances being processed, such as being dissolved, may be mixed together through a delivery pump, and this resulting mixture may be brought to an appropriate pressure and temperature through a heater pump that would cause the mixture to be placed under ultra-critical conditions and may then be delivered under this applied pressure into the cylindrical casing in the apparatus. While flowing through fluid flow paths, the mixture may be placed under the continuous ultra-critical conditions, under which the mixture may be crushed into ultrafine particles. The substances thus crushed into the ultrafine particles may then be exposed to ultrasonic waves, electromagnetic waves, laser rays, and the like so that they can react chemically or can be dissolved. Through this sequence of the steps described above, inter-molecule collision and dissolving may occur continuously, which may promote a chemical reaction. The substances that have thus been dissolved may be delivered to a cooler, and are then delivered to a gas-liquid separator where gas and liquid are separated, with the gas being processed to become harmless and extracted solvent in its liquid state being returned through return piping to a solvent vessel for recycling.

### Embodiment 7

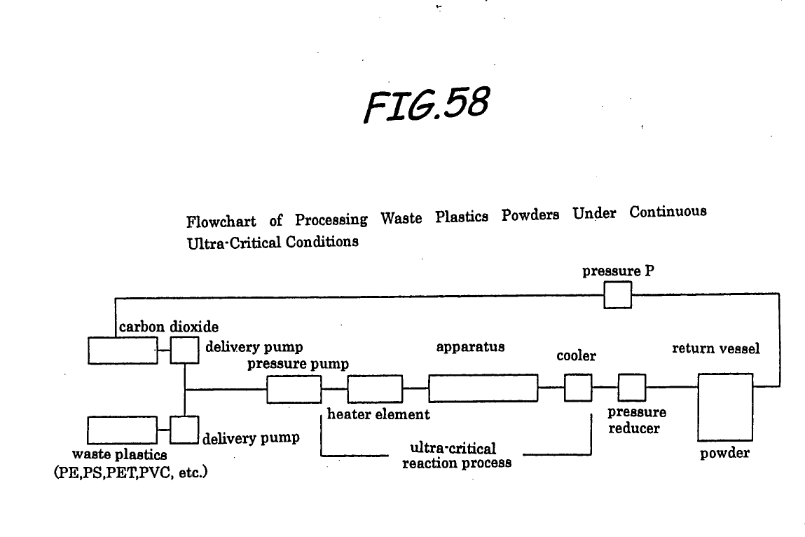

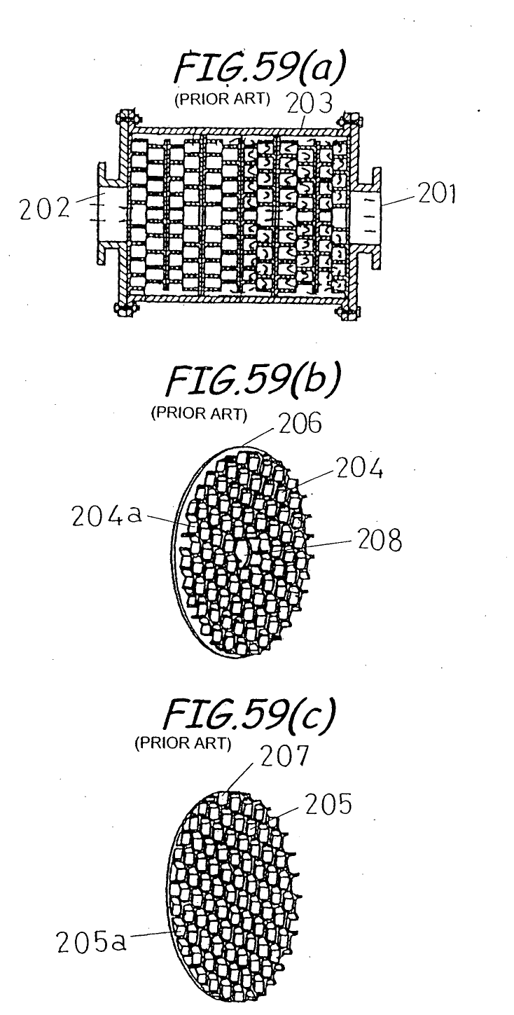

FIG. 58 is a block diagram illustrating a process of a method of the present invention that may be implemented by the apparatus described above in embodiments 1 through 4.

Waste plastics that have previously been crushed into gross particles, and carbon dioxide that is used as an extracted solvent and/or for oxidization reaction and hydrolysis, may be delivered together under applied pressure toward an inlet of the cylindrical casing through a delivery pump and pressure pump, and may then be heated by any heating elements such as a heater. At this moment, these substances should be placed under ultra-critical conditions for the carbon dioxide under which they should be maintained at a pressure of 7.38 MPa and a temperature of 31° C.

A fluid of this grossly crushed waste plastics that is placed under the ultra-critical conditions may then be delivered under an applied pressure into fluid flow paths from the inlet of the cylindrical casing, and then the fluid may flow under the applied pressure toward the outlet.

While flowing through the fluid flow paths within the cylindrical casing, the grossly crushed waste plastics may be placed under the continuous ultra-critical conditions under which they may be crushed into finer particles.

Those substances that have thus been processed and discharged from the outlet may be passed to a cooler and pressure reducer where the substances may be separated into plastics and powders. The powders may be collected into a return vessel, with the gas being returned for recycling.

In this embodiment, carbon dioxide is used as the extracted solvent and/or for an oxidization reaction and hydrolysis, but it should be understood that any type of extracted solvent other than that the carbon dioxide may also be used as long as such extracted solvent can be used when the substances are crushed into fine particles while they are placed under the critical and ultra-critical processing conditions.

The waste plastics that may be crushed into fine particles in this embodiment include polyethylene, polystyrene, polyethylene terephtalate and the like. It should be appreciated, however, that the present invention is not limited to such waste plastics, but other substances such as virgin materials, synthetic resin materials and the like may also be processed in the same manner as described above; specifically, may be crushed into fine particles while they are placed under the continuous critical or ultra-critical processing conditions.

For past years, waste plastics, virgin materials and synthetic resin materials in pellet forms were frozen, and then were crushed into powders. This is because there was no technology that permits such pellets to be crushed into powders at a normal temperature. But this freezing process was very costly.

When the apparatus of the present invention is used, however, waste plastics, and the like can be crushed into fine powders at lower costs because it eliminates a need for a freezing process.

When a conventional wet-type crushing machine is used to crush waste plastics, and the like into fine powders, it is very difficult to separate a product into powders and gas.

When the method steps shown in FIG. 58 and FIGS. 57(b), FIG. 57(c) are performed by the apparatus of the present invention, substances can be crushed into fine particles while they are placed under continuous critical and ultra-critical conditions, and resulting substances can be separated into powders and the gas continuously and easily.

It may be appreciated from the foregoing description that the apparatus according to any of the embodiments of the present invention provides both dry-type crushing functions and wet-type crushing functions.

### POSSIBLE INDUSTRIAL USES OF THE INVENTION

It may be appreciated from the foregoing description that the apparatus and method according to any of the embodiments of the present invention provides following advantages.

One advantage of the present invention is in that the apparatus includes a fluid flow path that permits a fluid of substances being processed, such as being mixed and/or crushed into fine particles, to have various actions and effects while the fluid flows through the fluid flow path, including compression by applied pressure followed by instantly explosive release from the compression, compression and dispersion followed by release from the compression and dispersion, production of turbulent flows within the fluid flow path, embracing pressure followed by release of pressure, and the like. These actions and effects may occur continuously so that the substances can be dissolved into fine particles, as explained by the dissipation theory.

This advantage has an accompanying effect that permits even fibrous substances to be crushed into fine particles having pure spherical forms.

Another advantage of the present invention is in that the apparatus includes a cylindrical casing, first and second structural members and/or frame members each having a plurality of cells thereon, each having its front side open, which may be made of composite metal materials such as combinations of carbon and copper, carbon and aluminum, carbon and magnesium, carbon and tungsten, and carbon and titanium oxide, and minerals such as ceramics and tourmaline that enable those mechanical members to provide catalyst actions.

This advantage has an accompanying effect that permits the first and second structural members to be made of natural or synthetic resins, on which the cells, each having its front side open and having a particular shape derived from a right-angled isosceles triangle, can be formed with higher precision.

A further advantage of the present invention is in that a plurality of magnets may be arranged on an outer circumference of the cylindrical casing such that they face opposite each other for providing N polarity and S polarity, wherein a fluid of substances being processed, such as being mixed and/or crushed, can be subdivided into finer particles under magnetic action, which can be mixed more uniformly.

Still another advantage of the present invention is in that the cylindrical casing has a divisible construction that permits the structural members and/or structural units to be mounted to or removed from the cylindrical casing for forming fluid flow paths within the cylindrical casing, by separating the cylindrical casing into two parts, and fitting the structural members or structural units in recesses provided on an inner circumferential wall of the cylindrical casing or removing them from the recesses. This permits easy assembling and reassembling, and disassembling for maintenance.

As the cylindrical casing has a construction that allows for easy mounting and dismounting of the structural members and/or structural units to and from the cylindrical casing for forming the fluid flow paths therein as described above, the structural members and/or structural units may be made of different types of materials so that they can form the fluid flow paths. Thus, substances can be mixed and/or crushed into fine particles under optimum processing conditions.

For example, a size, number, and shape of the cells, each having its front side open, which are provided on the structural members for forming the fluid flow paths, as well as a type of material for the structural members, may be changed, depending on particular properties and a mixture ratio of substances being processed. Thus, the substances may be mixed and/or crushed into fine particles under optimum conditions.

For industrial wastes, for example, these wastes may previously be crushed into gross particles in a form of a fluid, which may then be delivered under applied pressure, together with a particular gas, such as pure oxygen, into a fluid flow path within the cylindrical casing of the apparatus. Then, the fluid can have dispersing, colliding and swirling actions performed repeatedly, while flowing through the fluid flow paths formed by the cells. These actions can dissolve molecules bonded in the substances, thereby rendering the substances harmless.

In the apparatus according to any of the embodiments described above, the cylindrical casing, as well as the structural members forming the fluid flow paths, may be made of thermally conducting materials, such as copper, aluminum, carbon and other similar materials. In this case, the apparatus may be used as a heat exchanger, in which substances may be mixed and/or crushed into fine particles, while heat produced may be exchanged.

When a fluid of substances being processed, such as being mixed and/or crushed into fine particles, is delivered under applied pressure into the cylindrical casing, the fluid may flow through the fluid flow paths formed by the cells, each having its front side open, which are arranged to face opposite each other. As the fluid is flowing through the fluid flow path, the fluid may first flow into one cell, from which the fluid may then flow out to two cells, from which the fluid may then flow out to one cell, and so on. This is repeated, in which each time the fluid flows in and out, the fluid may be released instantly from pressure, thereby causing the fluid to explode outwardly, and may undergo strong compression. Thus, the substances may be crushed into ultrafine particles or molecules.

It may be understood from the foregoing description that the apparatus can be operated under critical or ultra-critical conditions for a particular type of substances being processed, such as substances that are hard to be dissolved, more specifically, industrial wastes, environment polluting substances and the like that might contain dioxins, under which such substances can be dissolved and rendered harmless. More specifically, by using the apparatus under the above conditions, a process of mixing the substances with a particular solvent, and crushing the substances into ultrafine particles or molecules, thereby promoting reaction dissolving, and dissolving, can be enhanced. Thus, a substance dissolving process can be improved. Furthermore, by using the apparatus in conjunction with an ultrasonic wave illuminating device, electromagnetic wave illuminating device, infrared ray illuminating device and/or far infrared ray illuminating device during the above sequence of operations, the process of crushing the substances into ultrafine particles or molecules and promoting a reaction dissolving can be enhanced further. Thus, a substance dissolving process can be improved much further.

Finally, by using the apparatus under the critical and ultra-critical conditions, any foodstuffs and raw medicinal substances can be processed continuously, and a process of deactivating, sterilizing, and deodorizing any enzymes or spores contained in those materials, can occur efficiently, safely and continuously. This includes a process of controlling a chemical reaction for chemical substances, generating chemical substances, dissolving chemical substances and the like.

What is claimed is:

1. An apparatus for mixing and/or crushing substances, comprising:
a casing defining a hollow interior and having an inlet at one end and an outlet at an opposite end;
first cells each having an open front side; and
second cells each having an open front side,
wherein said open front sides of said first cells face said open front sides of said second cells, with said first cells being placed in alternate positions with respect to said second cells, and with each of said first cells communicating with at least one of said second cells, whereby defined in said hollow interior is a fluid flow path that runs through said casing for allowing fluid to flow from said inlet towards said outlet,
wherein said open, front sides of said first cells each have a shape bounded by segments P-S, S-Q, Q-S2, S2-R, R-S1 and S1-P associated with a right-angled isosceles triangle having a hypotenuse, a first side and a second side, with
(i) S corresponding to any point on a line segment bisecting the right angle and the hypotenuse, other than the end points of this line segment,
(ii) P corresponding to any point on the first side, other than the end points of the first side,
(iii) Q corresponding to any point on the second side, other than the end points of the second side,
(iv) S1 corresponding to a point where segment P-S intersects the hypotenuse when segment P-S is turned about point P,
(v) S2 corresponding to a point where segment Q-S intersects the hypotenuse when segment Q-S is turned about point Q, and
(vi) R corresponding to the midpoint of the hypotenuse, and
wherein said open front sides of said second cells each have a shape bounded by segments P′-S′, S′-Q′, Q′-S2′, S2′-R, R-S1′ and S1′-P′ associated with the right-angled isosceles triangle, with
(i) S′ corresponding to any point on a line segment bisecting the right angle and the hypotenuse, other than the end points of this line segment,
(ii) P′ corresponding to any point on the first side, other than the end points of the first side,
(iii) Q′ corresponding to any point on the second side, other than the end points of the second side,
(iv) S1′ corresponding to a point where segment P′-S′ intersects the hypotenuse when segment P′-S′ is turned about point P′, and
(v) S2′ corresponding to a point where segment Q′-S′ intersects the hypotenuse when segment Q′-S′ is turned about point Q′.

2. The apparatus according to claim 1, wherein
P and P′ are the midpoint of the first side, and
Q and Q′ are the midpoint of the second side.

3. The apparatus according to claim 1, wherein
the area surrounded by segments P-S, S-Q, Q-S2, S2-R, R-S1 and S1-P is equal to one half the area of the right-angled isosceles triangle, and
the area surrounded by segments P′-S′, S′-Q′, Q′-S2′, S2′-R, R-S1′ and S1′-P′ is equal to one half the area of the right-angled isosceles triangle.

4. The apparatus according to claim 1, wherein
said fluid flow path extends in an axial direction of said casing or in a direction perpendicular to the axial direction of said casing.

5. The apparatus according to claim 1, wherein
said first cells are on a first structural member that is mountable within said casing,
said second cells are on a second structural member that is mountable within said casing, and
said fluid flow path is formed in an axial direction of said casing or in a direction perpendicular to the axial direction of said casing.

6. The apparatus according to claim 1, wherein
said first cells are on an inner peripheral wall of said casing, and
said second cells are on an outer peripheral wall of a structural mounted within said casing.

7. The apparatus according to claim 5, wherein
said casing is of a construction that allows said casing to be opened and closed, with said second structural member being removably mountable within said casing such that said second structural member can be removed from said casing when said casing is opened.

8. The apparatus according to claim 1, wherein
said first cells are on a first structural member comprising
(i) a plate member having said first cells on one side thereof, or
(ii) a plate member having said first cells on opposite sides thereof,
said second cells are on a second structural member comprising
(i) a plate member having said second cells on one side thereof, or
(ii) a plate member having said second cells on opposite sides thereof, and
said fluid flow path is formed in an axial direction of said casing or in a direction perpendicular to the axial direction of said casing.

9. The apparatus according to claim 8, wherein
said plate member having said first cells on opposite sides thereof comprises a plate member with said first cells on one of opposite sides thereof being behind said first cells on the other of opposite sides thereof and turned by a predetermined angle with regard to said first cells on the other of opposite sides thereof, and
said plate member having said second cells on opposite sides thereof comprises a plate member with said second cells on one of opposite sides thereof being behind said second cells on the other of opposite sides thereof and turned by a predetermined angle with regard to said second cells on the other of opposite sides thereof.

10. The apparatus according to claim 8, wherein
said plate member having said first cells on opposite sides thereof comprises a plate member with said first cells on one of opposite sides thereof being offset relative to said first cells on the other of opposite sides thereof, and
said plate member having said second cells on opposite sides thereof comprises a plate member with said second cells on one of opposite sides thereof being offset relative to said second cells on the other of opposite sides thereof.

11. The apparatus according to claim 8, wherein
said plate member having said first cells on opposite sides thereof comprises a plate member with said first cells on one of opposite sides thereof being offset relative to said first cells on the other of opposite sides thereof and turned by a predetermined angle with regard to said first cells on the other of opposite sides thereof, and
said plate member having said second cells on opposite sides thereof comprises a plate member with said second cells on one of opposite sides thereof being offset relative to said second cells on the other of opposite sides thereof and turned by a predetermined angle with regard to said second cells on the other of opposite sides thereof.

12. The apparatus according to claim 8, further comprising:
frame members arranged perpendicularly to the axial direction of said casing, said frame members having openings arranged in a honeycomb configuration on an upstream side and a downstream side of said fluid flow path, with said openings providing a communicating path extending in a direction of said casing, and with any two adjacent said frame members being arranged to face opposite each other such that said openings of one of said any two adjacent said frame members are placed in alternate positions with regard to said openings of the other of said any two adjacent said frame members.

13. The apparatus according to claim 8, wherein
said first cells are on a first plate member that is mountable within said casing,
said second cells are on a second plate member that is mountable within said casing, and
said casing is of a construction that allows said casing to be opened and closed, with said first and second plate members being removably mountable within said casing such that said first and second plate members can be removed from said casing when said casing is opened.

14. The apparatus according to claim 12, wherein
said first cells are on a first plate member that is mountable within said casing,
said second cells are on a second plate member that is mountable within said casing, and
said casing is of a construction that allows said casing to be opened and closed, with said first and second plate members and said frame members being removably mountable within said casing such that said first and second plate members and said frame members can be removed from said casing when said casing is opened.

15. The apparatus according to claim 1, wherein
each of said open front sides of said first cells is of the same shape as each of said open front sides of said second cells.

16. The apparatus according to claim 1, wherein
said first cells are arranged in a honeycomb configuration, and/or
said second cells are arranged in a honeycomb configuration.

17. The apparatus according to claim 1, further comprising:
an inlet space on an upstream side of said fluid flow path and an outlet space on a downstream of said fluid flow path, with said inlet space having a frustoconical shape having a diameter that increases away from said inlet, and said outlet space having a frustoconical shape having a diameter that decreases toward said outlet.

18. The apparatus according to claim 17, further comprising:
a first structural member, having the frustoconical shape of said inlet space, in said inlet space for forming a flow path between an inner peripheral wall defining said inner space and an outer peripheral wall of said first structural member, and
a second structural member, having the frustoconical shape of said outlet space, in said outlet space for forming a flow path between an inner peripheral wall defining said outer space and an outer peripheral wall of said second structural member.

19. The apparatus according to claim 1, wherein
said first cells and/or said second cells are of any material selected from the group consisting of carbons, composite metal materials including carbons combined with other metals, ceramics, and minerals.

20. The apparatus according to claim 1, wherein
said casing is of any material selected from the group consisting of carbons, composite metal materials including carbons combined with other metals, ceramics, and minerals.

21. The apparatus according to claim 1, wherein
said first cells and/or said second cells are of any material selected from the group consisting of resins and synthetic resins.

22. The apparatus according to claim 1, wherein
said casing is of any material selected from the group consisting of resins and synthetic resins.

23. The apparatus according to claim 1, further comprising:
a magnet mounted on an outer peripheral surface of said casing.

24. The apparatus according to claim 1, further comprising:
at least one of an ultrasonic illuminating device, an electromagnetic wave illuminating device, a high frequency wave illuminating device and a laser beam illuminating device, linked to an upstream and/or downstream side of said casing.

25. The apparatus according to claim 1, further comprising:
an infeed device, linked to an upstream and/or downstream side of said casing, for feeding into said casing different types of substances.

26. A method for mixing and/or crushing substances by using the apparatus as defined in claim 1, comprising:
introducing a fluid, having substances mixed therein, under applied pressure through said inlet of said casing; and
causing said fluid to flow through said fluid flow path from said inlet towards said outlet of said casing.

27. The method according to claim 26, wherein
causing said fluid to flow through said fluid flow path results in said substances being crushed into fine particles.

28. A method for mixing and/or crushing substances by using the apparatus as defined in claim 1, comprising:
continuously introducing a fluid, having substances mixed therein, under applied pressure through said inlet of said casing;
continuously placing said fluid under critical or ultra-critical conditions; and
while under said critical or ultra-critical conditions, causing said fluid to flow through said fluid flow path from said inlet towards said outlet of said casing.

29. The method according to claim 28, wherein
causing said fluid to flow through said fluid flow path results in said substances being crushed into fine particles.
